(19) United States
(12) Patent Application Publication
(10) Pub. No.: US 2023/0201245 A1
(43) Pub. Date: Jun. 29, 2023

(54) COMPOSITIONS AND METHODS FOR TREATING VIRUS INFECTION

(71) Applicants: CZ BIOHUB SF, LLC, San Francisco, CA (US); THE BOARD OF TRUSTEES OF THE LELAND STANFORD JUNIOR UNIVERSITY, Stanford, CA (US); THE REGENTS OF THE UNIVERSITY OF CALIFORNIA, Oakland, CA (US)

(72) Inventors: Judith Frydman, Stanford, CA (US); Raul Andino-Pavlovsky, Oakland, CA (US); Ranen Aviner, Stanford, CA (US)

(21) Appl. No.: 17/928,496

(22) PCT Filed: Jun. 4, 2021
(86) PCT No.: PCT/US21/35824

§ 371 Date: Nov. 29, 2022

Related U.S. Application Data
(60) Provisional application No. 63/035,185, filed on Jun. 5, 2020.

(51) Int. Cl. A61K 31/713; A61K 31/343; A61K 45/06; C12N 15/63; A61K 31/4439

(52) U.S. Cl. CPC A61K 31/713; A61K 31/343; A61K 45/06; C12N 15/63; A61K 31/4439; C12N 15/1137

(57) ABSTRACT

The present disclosure provides materials and methods for treating or preventing an infection from a virus such as an RNA virus, inhibiting replication of a vims in a cell, inhibiting translation of viral proteins in a cell infected with a virus, inhibiting prolyl hydroxylation in a cell infected with a vims, preventing or inhibiting viral-induced remodeling of a polysome in a cell, and identifying a polysome-associated protein in a cell infected by a virus.

### CROSS-REFERENCE TO RELATED APPLICATIONS

The present application claims priority to U.S. Provisional Patent Application No. 63/035,185 filed Jun. 5, 2020 the entirety of which is incorporated by reference herein.

### STATEMENT REGARDING FEDERALLY SPONSORED RESEARCH

The invention was made with Government support under Grant Nos. AI127447, R01 AI36178, AI40085, and P01 AI091575 awarded by National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55336_Seqlisting.txt”, which was created on Jun. 2, 2021 and is 2,826 bytes in size. The subject matter of the Sequence Listing is incorporated herein in its entirety by reference.

### BACKGROUND

Viruses are obligate intracellular parasites that are unconditionally dependent on their hosts for protein synthesis and have therefore evolved complex mechanisms to divert host resources and control the translational landscape. Production of viral progeny often involves unique biosynthetic challenges. For example, positive-strand RNA viruses are translated as a long multifunctional, multidomain polyprotein that requires further cleavage into individual subunits, thus increasing its propensity for misfolding and aggregation (reviewed in (Aviner and Frydman, 2020)). Furthermore, viral infection is commonly associated with shutoff of host translation to curtail antiviral responses and minimize competition over limiting resources. This is achieved primarily by inactivation of the eIF4F cap-binding complex, a key regulator of 40S ribosomal subunit recruitment. Under these conditions, viruses employ noncanonical strategies to allow continued production of viral proteins, e.g., translation initiation from internal ribosome entry sites (IRES) (reviewed in (Stern-Ginossar et al., 2019)). Therefore, ribosomes in infected cells are not only a platform for synthesis, folding and assembly of viral proteins; they are also a nexus that integrates opposing anti- and pro-viral signals propagated by the host and the virus, respectively, to either promote an antiviral state or remodel the environment to allow preferential translation of viral over cellular RNA.

Rearrangement of ribosomal components or ribosome-associated networks, generating subpopulations sometimes referred to as “specialized ribosomes”, has been reported across normal and pathogenic states and involves association or dissociation of core ribosomal proteins, enzymatic modification of ribosomal RNA or proteins, and selective recruitment of RNA-binding factors and molecular chaperones (reviewed in (Genuth and Barna, 2018)). Several studies have reported on the use of mass-spectrometry to study ribosome composition (Aviner et al., 2017; Imami et al., 2018; Simsek et al., 2017; Yoshikawa et al., 2018), but a systematic survey of polysomes in viral infection has not yet been attempted. Nevertheless, some ribosomal proteins have already been implicated in the regulation of viral translation. For example, 40S subunit RACK1 and eS25 are dispensable for cap-dependent initiation but critical for IRES initiation in multiple viruses (Landry et al., 2009; Majzoub et al., 2014), and 60S subunit eL40 and P1/P2 facilitate translation initiation and elongation of specific viruses but are not required for bulk protein synthesis (Campos et al., 2017; Lee et al., 2013). Some viruses even encode for ribosomal proteins that can become incorporated into host ribosomes (Mizuno et al., 2019). Furthermore, multiple RNA-binding factors were found to interact with viral RNA and either stimulate or inhibit translation (Ooi et al., 2019), while molecular chaperones e.g. Hsp70 heat shock proteins were shown to bind viral nascent chains and guide their folding and assembly (Aviner and Frydman, 2020). Recently, attempts have been made to develop modulators of protein folding as antivirals, including small molecule inhibitors against Hsp70 and Hsp90, as well as protein disulfide and peptidylprolyl isomerases, which show broad-spectrum activity against a wide range of viruses (Aviner and Frydman, 2020). These chaperones are involved in multiple steps of the viral life cycle, including entry, protein production, genome replication and virion assembly, rendering many viruses hypersensitive to their inhibition at non-toxic concentrations. Identifying additional cellular targets that are crucial for viral biogenesis could therefore help expand the pool of candidate compounds and provide hope for new modes of prophylactic and therapeutic antiviral interventions.

### SUMMARY OF THE INVENTION

In various aspects, the present disclosure provides methods for treating or preventing virus infections. In one embodiment, a method of treating or preventing an infection from a RNA virus in a subject is provided comprising the steps of administering a therapeutic agent, wherein said therapeutic agent is an inhibitor of (i) viral protein translation and/or (ii) an inhibitor of viral replication to the subject, wherein said inhibitor is capable of inhibiting the function of one or more eukaryotic initiation factors associated with a viral polysome. In various embodiments, the one or more eukaryotic initiation factor is selected from the group consisting of eukaryotic initiation factor 4A1 (eIF4A1) and eukaryotic initiation factor 4A2 (eIF4A2). Other RNA helicases are also contemplated, including but not limited to DDX3X/Y, DDX46, DDX55, AQR, DDX23, DDX18, DDX21, DDX50, DDX39B, DHX9, DDX6.

In another embodiment, the present disclosure provides a method of treating or preventing an infection from a RNA virus in a subject comprising the steps of administering an inhibitor of (i) viral protein folding and assembly and/or (ii) an inhibitor of viral replication to the subject, wherein said inhibitor is capable of inhibiting the function of one or more collagen prolyl hydroxylases and/or one or more collagen prolyl hydroxylase coenzymes. In various embodiments, the one or more collagen prolyl hydroxylase is a collagen prolyl 3-hydroxylase (CP3H) selected from the group consisting of prolyl 3-hydroxylase 1 (P3H1), prolyl 3-hydroxylase 2 (P3H2), and prolyl 3-hydroxylase 3 (P3H3). Non-specific prolyl hydroxylase inhibitors are also contemplated herein.

In still another embodiment, the one or more collagen prolyl hydroxylase is a collagen prolyl 4-hydroxylase (CP4H) selected from the group consisting of prolyl 4-hydroxylase subunit alpha-1 (P4HA1), prolyl 4-hydroxylase subunit alpha-2 (P4HA2) and prolyl 4-hydroxylase subunit alpha-3 (P4HA3). In other embodiments, the one or more collagen prolyl hydroxylase coenzymes is selected from the group consisting of cartilage associated protein (CRTAP) and synaptonemal complex 65 (Sc65/P3H4).

The present disclosure further provides, in some embodiments, an aforementioned method wherein said RNA virus is a flavivirus. In some embodiments, the flavivirus is selected from the group consisting of Zika virus, Dengue virus West Nile, Yellow Fever and Japanese Encephalitis.

In yet other embodiments of the present disclosure, an aforementioned method is provided wherein said inhibitor is selected from the group consisting of a small molecule inhibitor, an antibody or binding fragment thereof, an oligonucleotide, and a vector encoding an oligonucleotide. In some embodiments, the inhibitor is an oligonucleotide selected from the group consisting of a small inhibitory RNA (siRNA), a microRNA (miRNA), and a short hairpin RNA (shRNA). In one embodiment, the inhibitor is a vector that encodes an oligonucleotide of claim 10, a nuclease and/or a guide RNA (gRNA).

In one embodiment, an aforementioned method is provided wherein said eukaryotic initiation factor is eukaryotic initiation factor 4A1 (eIF4A1). In a related embodiment, the inhibitor is rocalgamide (RocA) or a flavagline.

In yet another embodiment, an aforementioned method is provided wherein the inhibitor is pythiDC. In other embodiments, the inhibitor is NOG, 24PDC, 25PDC, DHB, N-acylsulfonamide, 2-heterocyclic glycinamide, bipy, bipy45′DC, bipy55′DC, alpha-ketoglutarate, deferoxamine, pyoxDC, pyimDC. In other embodiments, the inhibitor is EDHB (Ethyl 3,4-dihydroxybenzoate).

In still other embodiments, an aforementioned is provided further comprising administering a second therapeutic agent. In some embodiments, the second therapeutic agent is selected from the group consisting of an anti-viral small molecule, an inhibitor of a chaperone protein, small molecule inhibitor, an antibody or binding fragment thereof, an oligonucleotide, and a vector encoding an oligonucleotide. In still other embodiments, the oligonucleotide is a small inhibitory RNA (siRNA), a microRNA (miRNA), and a short hairpin RNA (shRNA). In one embodiment, the vector encodes an oligonucleotide of claim 17, a nuclease and/or a guide RNA (gRNA). In yet other embodiments, the second therapeutic agent inhibits the function of a Hsp70 chaperone or an Hsp90 chaperone, or a cofactor of Hsp70 or Hsp90 or Hsp47. In various embodiments, the second therapeutic agent is selected from the group consisting of JG40, JG345, Apoptozole, PIFITHRIN-Mu, 115-7c, MALS-101 (Hsp70), AK778, Co1003, BMS-986263/NDL02-s0201 (Hsp47); geldanamycin, radicicol, derrubone, ganetespib, celastrol, novobiocin, VER49009, AT13387, PU3, PUH71, PUWS13 (Hsp90); AUY922, (Hsp90) VER155008, JG98, JG13, JG48, YM-01, YM-08 MKT-077, and PES-CI (Hsp70). In still other embodiments, the second therapeutic agent is selected from the group consisting of an inhibitor is capable of inhibiting the function of one or more eukaryotic initiation factors associated with a viral polysome, one or more collagen prolyl hydroxylases and/or one or more collagen prolyl hydroxylase coenzymes.

The present disclosure also contemplates preventing viral infections. Thus, in some embodiments, an aforementioned is provided wherein the therapeutic agent is administered prior to exposure and/or infection of said RNA virus. In another embodiment, the method of preventing further comprises administering a second therapeutic agent, wherein said second therapeutic agent is administered. In some embodiments, viral RNA polymerase inhibitors or viral protease inhibitors or viral entry inhibitors are contemplated.

The present disclosure provides, in various embodiments, an aforementioned method wherein the subject is human.

The present disclosure also provides, in various embodiments, additional methods. For example, in one embodiment, the present disclosure provides a method of inhibiting replication of a RNA virus in a cell comprising the steps of administering a therapeutic agent, wherein said therapeutic agent is an inhibitor of (i) viral protein translation and/or (ii) an inhibitor of viral replication to the subject, wherein said inhibitor is capable of (i) inhibiting the function of one or more eukaryotic initiation factors associated with a viral polysome or (ii) inhibiting the function of one or more collagen prolyl hydroxylases and/or one or more collagen prolyl hydroxylase coenzymes.

In another embodiment, the present disclosure provides a method of inhibiting translation of viral proteins in a cell infected with a RNA virus comprising the steps of administering a therapeutic agent, wherein said therapeutic agent is an inhibitor of (i) viral protein translation and/or (ii) an inhibitor of viral replication to the subject, wherein said inhibitor is capable of (i) inhibiting the function of one or more eukaryotic initiation factors associated with a viral polysome or (ii) inhibiting the function of one or more collagen prolyl hydroxylases and/or one or more collagen prolyl hydroxylase coenzymes.

In still another embodiment, a method of inhibiting prolyl hydroxylation in a cell infected with a RNA virus is provided comprising the steps of administering a therapeutic agent, wherein said therapeutic agent is an inhibitor of (i) viral protein translation and/or (ii) an inhibitor of viral replication to the subject, wherein said inhibitor is capable of inhibiting the function of one or more collagen prolyl hydroxylases and/or one or more collagen prolyl hydroxylase coenzymes.

In yet another embodiment, the present disclosure provides a method of preventing or inhibiting viral-induced remodeling of a polysome in a cell comprising the steps of administering a therapeutic agent, wherein said therapeutic agent is an inhibitor of (i) viral protein translation and/or (ii) an inhibitor of viral replication to the subject, wherein said inhibitor is capable of (i) inhibiting the function of one or more eukaryotic initiation factors associated with a viral polysome or (ii) inhibiting the function of one or more collagen prolyl hydroxylases and/or one or more collagen prolyl hydroxylase coenzymes.

In some embodiments, a second therapeutic agent is administered.

The present disclosure also provides a method of identifying a polysome-associated protein in a cell infected by a virus comprising the steps of: (a) obtaining a sample of cells; (b) infecting said cells of (a) with a virus; (c) isolating polysomes from infected cells of (b); and (d) analyzing protein composition of said polysomes. In one embodiment, the protein composition is analyzed by mass spectrometry. In still other embodiments, the method further comprises the steps of comparing the composition of polysomes of and identifying proteins distinguishing polysomes from virus infected cells.

### BRIEF DESCRIPTION OF THE DRAWINGS

FIG. 1 shows that RNA viruses extensively remodel polysome composition during infection. (FIG. 1A-B) Schematic representation of viral genome and polyprotein organization for poliovirus (PV) (FIG. 1A) and Zika (ZIKV) or dengue (DENV) (FIG. 1B). Green and pink/purple represent cytosolic and ER membrane-associated domains, respectively. (FIG. 1C) Infection with either PV, ZIKV or DENV is associated with a shutoff of host protein synthesis and selective production of viral proteins. Huh7 cells were infected with either of the three viruses at a multiplicity of infection (MOI) of 5. At the indicated timepoints, puromycin was added to the tissue culture medium to label nascent chains and cell lysates were subjected to immunoblot. (FIG. 1D) Cells were infected with PV, ZIKV or DENV at MOI=5 for 4, 36 and 48 h, respectively, and lysates were fractionated on 10-50% sucrose gradients with continuous monitoring of OD254 rRNA traces (gray). Co-sedimentation of nascent chains (orange) and plus-strand viral RNA (blue) was probed by puromycin labeling and qPCR, respectively. (FIG. 1E) Experimental design for probing the composition of translating polysomes in infected cells. Huh7 cells were infected in parallel with PV, ZIKV or DENV at MOI=5. Cell lysates were fractionated on 10-50% sucrose gradients, and polysomes were extracted by a second round of ultracentrifugation on a 66% sucrose cushion. Recovered proteins were digested and subjected to liquid chromatography tandem mass-spectrometry analysis (LC-MS/MS). (FIG. 1F) Main functional categories represented in the polysome interactors dataset across all samples. Spike length is proportional to protein amounts quantified by MS. (FIG. 1G) Samples for MS analysis were collected at timepoints reflecting similar biosynthetic steps for each of the three viruses i.e. before, during and after shutoff of translation. (FIG. 1H) Polysome composition is gradually and extensively remodeled during infection with PV, ZIKV and DENV. Shown is unsupervised hierarchical clustering of Spearman's correlation coefficients across all polysome interactor samples.

FIG. 2 shows infection is associated with pan-viral and virus-specific rearrangement of ribosome-associated biosynthetic pathways. (FIG. 2A) Core ribosome composition remains mostly unchanged during infection with PV, ZIKV and DENV. Volcano plot shows pairwise comparisons of ribosomal protein levels in polysome fractions in infected versus mock-infected cells. (FIG. 2B) ZIKV and DENV share common polysome dynamics that are distinct from PV. Shown is a principal component analysis (PCA) of non-ribosomal proteins in polysome fractions. (FIG. 2C-D) Temporal patterns of polysome interactions reveal distinct categories of interactors. Unsupervised hierarchical clustering of Z-scored polysome interactors (right), compared side by side with their steady-state levels (left) (FIG. 2C) with line plot examples of each of the 6 major temporal patterns identified (FIG. 2D). Line plots show MS intensity for all three replicates, with known viral interactors indicated for each host protein. IRES, Internal ribosome entry site; P1 and C, viral capsid proteins; E, viral envelope protein; Ribo., ribosome. (FIG. 2E) Polysomes translating cytosolic PV and ER-associated ZIKV/DENV show compositional dynamics consistent with biosynthetic requirements. Gene Ontology (GO) annotations with manual validation were used to calculate an average fold change from mock for all polysome interactors detected in each category (heatmap), with cellular compartment and ANOVA p-value indicated for each.

FIG. 3 shows RNA surveillance factors are depleted from polysomes by viral protease cleavage or subgenomic sequestration. (FIG. 3A) STRING interaction network of polysome interactors involved in RNA surveillance and quality control that show statistically significant changes during infection with either PV, ZIKV or DENV. Node size is proportional to the maximum level of each protein in the polysome interactions dataset. (FIG. 3B) G3BP1 and interacting proteins are depleted off polysomes during infection with PV, ZIKV or DENV. Line plots show MS intensity for all three replicates. (FIG. 3C) G3BP1 is cleaved in PV−, but not ZIKV− or DENV−, infected cells. (FIG. 3D) Host factors known to be cleaved by PV proteases are depleted off polysomes during infection. Volcano plot shows pairwise comparisons of polysome interactors in infected versus mock-infected cells. (FIG. 3E) Polysome interactors depleted during ZIKV infection include high affinity sfRNA binding proteins involved in RNA surveillance. Scatter plot shows pairwise comparisons of polysomes interactors (x axis) and vRNA-interacting proteins detected by RNA pulldown from ZIKV-infected cells (y-axis). Inset shows violin plots for polysome interactors depleted from or recruited to polysomes by more than 2 fold in ZIKV-infected cells. (FIG. 3F) Most 3′UTR-containing ZIKV RNA does not associate with polysomes. Cells were infected with ZIKV at MOI=5 for 24 h, and lysates were fractionated on sucrose gradients. Plus strand vRNA was detected by qPCR. (FIG. 3G) Two models explain the depletion of polysome interactors during infection: cleavage by viral protease or sequestration by viral subgenomic fragments.

FIG. 4 shows eIF4A1 regulates translation and replication in ZIKV infection. (FIG. 4A) STRING interaction network of polysome interactors involved in translation that show statistically significant changes during infection with either PV, ZIKV or DENV. Node size is proportional to the maximum level of each protein in the polysome interactions dataset. (FIG. 4B) eIF4A1 is recruited to polysomes translating ZIKV and DENV, while other translation initiation factors are depleted across all three viruses. Line plots show means±SD of the indicated factors. (FIG. 4C) eIF4A1 inhibition reduces virus production in ZIKV and DENV infected cells. Cells were infected with PV, ZIKV or DENV in the presence or absence of increasing concentrations of RocA, a specific eIF4A1 inhibitor, and virus production was measured at the indicated timepoints using plaque assays. Shown are means±SD of 3 independent repeats. (FIG. 4D) eIF4A1 inhibition blocks translation initiation on ZIKV RNA. Cells were infected with ZIKV for 24 h then treated with 30 nM RocA for 30 min. Lysates were fractionated on sucrose gradients and GAPDH mRNA, ZIKV plus-strand RNA levels were quantified in each fraction by qPCR. Shown are means±SD of 3 independent repeats. (FIG. 4E-G) RocA inhibits both translation and replication in ZIKV infection. Immunoblot of ZIKV-infected cells treated at 24 hpi with RocA, CHX, Puro or CHX+Puro and harvested at 36 hpi (E). Quantification of (+)vRNA and viral particle production by qPCR and plaque assays, respectively (F). In vitro replication assay with clickable uridine analog (5EU) in the absence or presence of RocA and NS5 inhibitor 2′CMA (G).

FIG. 5 shows molecular chaperones regulate co-translational aggregation, ER targeting and nascent chain processing in infected cells. STRING interaction network of polysome associated cytosolic proteostasis factors that show statistically significant changes during infection with either PV, ZIKV or DENV. Node size is proportional to the maximum level of each protein in the polysome interactions dataset. (FIG. 5B) Depletion of cytosolic chaperones impairs virus production. Cells were transduced with lentiviruses and puromycin selected to generate stable KDs, then infected with PV, ZIKV or DENV. Virus production was measured using plaque assays. Shown are means±SD of 3 independent repeats. (FIG. 5C) Chemical structure of Hsp70 inhibitors JG40 and JG345. (FIG. 5D-E) Hsp70 inhibitors show differential virus specificity. Cells were infected with PV, ZIKV or DENV (FIG. 5D) or with PV alone (E), Hsp70 inhibitors were added upon removal of inoculum, and virus production was measured using plaque assay. Shown are means±SD of 3 independent repeats. (FIG. 5F) Hsp70 protects PV nascent chains from co-translational aggregation. Cells were infected with PV and Hsp70 inhibitors were added upon removal of inoculum. At 3.5 hpi, puromycin was added to label nascent chains and lysates were subjected to immunoblot analysis. (FIG. 5G) STRING interaction network of polysome associated ER proteostasis factors, similar to A. (FIG. 5H) Depletion of ER chaperones impairs ZIKV and DENV production. Cells were transduced with lentiviruses and puromycin selected to generate stable KDs, then infected with ZIKV or DENV. Virus production was measured using plaque assays. Shown are means±SD of 3 independent repeats.

FIG. 6 shows Collagen prolyl hydroxylases promote ZIKV and DENV biogenesis. (FIG. 6A-B) Peptidylprolyl modifying enzymes are recruited to polysomes translating ZIKV and DENV, but not PV. Shown are STRING network analysis (FIG. 6A) and line plots of MS intensity data for all three replicates (FIG. 6B). (FIG. 6C) Collagen prolyl-4-hydroxylase (P4HA1) co-sediments with polysomes in cells infected with ZIKV and DENV, but not PV. Lysates of infected cells were fractionated on 10-50% sucrose gradients. rRNA absorbance was monitored continuously (gray profiles) and fractions were incubated with puromycin to label nascent chains (orange) and subjected to immunoblot analysis. (FIG. 6D) Depletion of collagen prolyl-4-hydroxylases impairs ZIKV and DENV production. Cells were transduced with lentiviruses and puromycin selected to generate stable KDs, then infected with ZIKV or DENV. Virus production was measured using plaque assays. Shown are means±SD of 3 independent repeats. (FIG. 6E) Small molecule inhibitor against collagen prolyl hydroxylases, but not other prolyl hydroxylases, impair ZIKV and DENV production. Effects of inhibitors on viral titers and cell viability were measured using plaque assays.

FIG. 7 shows prolyl hydroxylation modulates stability of ZIKV proteins. (FIG. 7A) Proline hydroxylation is required for polysome remodeling. WT and cP4H-KD cells were infected with ZIKV and polysomes were isolated for MS analysis as described in 1E. Scatter plots show pairwise comparisons of polysome interactors between infected and mock-infected WT and cP4H-KD cells; r=Spearman's correlation coefficient. (FIG. 7B) MS data was searched for evidence of proline hydroxylation of ZIKV proteins. Each bubble represents a modified site, and its size is proportional to the ratio of hydroxyproline (hyp) modified/unmodified in WT (black) and cP4H-KD (orange). Discrete proteins produced by cleavage of the polyprotein are indicated on top. Bar graph shows similar levels of unmodified peptides spanning Proline 1375. (FIG. 7C) Proline position in respect to NS2B transmembrane domains (TMD) 1 and 2 is conserved in flaviviruses. (FIG. 7D) ZIKV protein stoichiometry is altered upon cP4H depletion. ZIKV peptides detected by MS analysis of polysomes in WT and cP4H-KD cells are plotted as cumulative distribution (top) or bubbles (bottom) ordered by position on the ZIKV polyprotein, with bubble size proportional to peptide intensity. Bar graph shows the ratio of E to NS5 peptides in WT and cP4H-KD cells. (FIG. 7E) cP4H depletion is associated with reduced accumulation of E. Cells were infected with ZIKV for 24 or 36 h, pulse labeled with radioactive amino acids and chases for 6 h. Shown are autoradiogram and immunoblots. (FIG. 7F) E is rapidly degraded in the absence of cP4H. Cells were infected with ZIKV for 24 h and MG132 or CHX were added for 12 h. (FIG. 7G) Proposed function of cP4H in ZIKV infection. In WT cells, ZIKV is co-translationally modified by cP4H, promoting correct topology and stable protein products. In cP4H-KD cells, ZIKV is not modified co-translationally and parts of the polypeptide do not assume correct topology and become degraded.

### DETAILED DESCRIPTION

Understanding how pathogenic viruses interact with the cellular environment to facilitate viral protein production and dampen the innate immune response is critical to developing new antiviral strategies. The present disclosure provides a proteomic analysis of isolated polysomes to study co-translational virus-host interactions at multiple timepoints of infection with polio, zika or dengue viruses. This revealed extensive remodeling of polysome associated networks, including antiviral RNA sensing, translation initiation, nascent chain folding and polyprotein processing. As described herein, helicase eIF4A1 is specifically involved in translation and replication of Zika and Dengue, and Hsp70 chaperones protect polio from co-translational aggregation. The present disclosure further provides that collagen prolyl hydroxylases modify viral nascent chains and that this modification promotes zika and dengue infection by stabilizing viral proteins. Taken together, the present disclosure demonstrates the importance of polysome interactome analysis in uncovering and targeting new cellular pathways involved in viral pathogenesis to prevent infection.

The present disclosure is the first proteomic analysis of polysome dynamics during infection with three positive-strand RNA viruses. Although many individual host factors were previously shown to modulate viral protein synthesis and assembly, this disclosure is the first to characterize the virus-induced translational state using an unbiased systems-level approach, generating new insights into viral pathogenesis. The present disclosure provides that infection with either PV, ZIKV or DENV is associated not only with a shutoff of global translation but also unprecedented remodeling of polysome composition that is not driven or reflected by changes in steady-state protein levels. Interactors recruited to polysomes during infection showed the expected subcellular partitioning between the cytosolic PV and ER-associated ZIKV and DENV, and encompassed a wide range of functions from regulation of translation initiation and antiviral RNA sensing to polypeptide folding, enzymatic modification and ER-associated degradation. One striking advantage of the present disclosure over other interactome studies is that it reports not only on interactors that bind viral polysomes but also interactors that are displaced in response to infection, providing an added dimension of directionality.

PV, ZIKV and DENV all suppress cap-dependent translation in Huh7 cells, but only PV is known to initiate cap-independent translation from a bonafide IRES. While the absence of an IRES in ZIKV and DENV genomes suggests cap dependence, as confirmed by in vitro assays (Sanford et al., 2019), the present disclosure shows that flaviviral genomes remain engaged with polysomes even after global shutoff of translation. Furthermore, almost all canonical initiation factors required for cap-dependent translation, including eIF4G1, eIF3 and PABPC1, were depleted off polysomes during infection with any of the viruses. In contrast, multiple RNA-binding proteins known to activate PV IRES were specifically recruited to PV polysomes by orders of magnitude. Interestingly, the top three most recruited ITAFs, namely GARS, CSDE1 and STRAP are not critical for in vitro translation of PV (Sweeney et al., 2014), suggesting that viral translation in intact cells involves additional challenges. In the absence of an IRES, ZIKV and DENV can initiate translation using other RNA structure-based strategies e.g. 3′ UTR cap-independent translation elements (3′CITE), which recruit the 60S subunit in some plant viruses (reviewed in (Jaafar and Kieft, 2019)). This is supported by the observation that translation of a reporter flanked by flaviviral UTRs is inhibited by oligonucleotides that bind to the 3′UTR (Fan et al., 2011). Alternatively, spatial compartmentalization of the translation machinery, as reported for other viruses (e.g. reovirus, (Desmet et al., 2014)), could promote efficient translation by increasing the local concentration of factors.

Regardless of the mechanism of initiation on ZIKV and DENV, the present disclosure provides that it is hyperdependent on helicase eIF4A1. Inhibition of eIF4A1 by RocA strongly reduced production of both ZIKV and DENV, with only minor effects on PV. Other eIF4A1 inhibitors e.g. silvestrol and pateamine A were recently shown to have antiviral activity against ebola (Biedenkopf et al., 2017), chikungunya (Henss et al., 2018), influenza (Slaine et al., 2017) and ZIKV (Elgner et al., 2018), but using RocA could have additional advantages as it irreversibly clamps eIF4A1 to its target RNA. Because genome circularization and ribosome scanning are mutually exclusive in ZIKV and DENV (Sanford et al., 2019), and considering how critical genome circularization is for replication, any drug that immobilizes host proteins on the viral genome could potentially interfere with multiple steps in the viral lifecycle. Indeed, the present disclosure shows that stabilization of the translation-competent conformation by either RocA-mediated eIF4A1 clamping or cycloheximide-induced arrest of elongating ribosomes leads to reduced viral replication. Interestingly, hnRNPD (AUF1), DHX9 (RHA) and ILF2/3, which are known to promote flavivirus replication by triggering genome circularization (Friedrich et al., 2018; Isken et al., 2007), were recruited to PV polysomes but depleted from ZIKV and DENV polysomes, supporting a complex regulation of the switch between translation and replication in flaviviral infection. Thus, the data herein can be used to identify RNA-binding proteins that, when targeted, may inhibit IRES activation in PV or translation initiation and genome replication in ZIKV and DENV.

Synthesis and folding of the long, multidomain viral polyprotein involve unique challenges. One such challenge is associated with capsids of unenveloped viruses, which are particularly sensitive to misassembly as they need to form oligomeric structures both rigid enough to protect the enclosed genome and flexible enough to allow its timely release (Aviner and Frydman, 2020). Misassembly of some cellular complexes has been linked to aggregation when subunits fail to engage their partners co-translationally (Shiber et al., 2018), and failure of ribosome quality control to clear stalled ribosomes is also associated with nascent chain aggregation (Choe et al., 2016). Using puromycylation, it is shown that cytosolic Hsp70 protects nascent PV capsids from their innate propensity to oligomerize and aggregate. PV synthesis likely taxes the Hsp70 system, as co-translational aggregation was detected around the onset of global translation shutoff even without Hsp70 inhibitors. Although both Hsp70 inhibitors tested bind to the same allosteric site on Hsp70 and block nucleotide exchange factor (NEF) interactions, only one had noticeable effects on either co-translational aggregation or virion production, suggesting that characterization of inhibitor specificity may lead to discovery of additional co-chaperones involved in viral protein folding.

Other than chaperones, nascent chain modifying enzymes associated with viral polysomes, including proline modifying enzymes. Inhibitors of peptidylprolyl isomerases have been explored as broad-spectrum antivirals, as this class of enzymes is critical for many viruses (Peel and Scribner, 2013). Proline hydroxylation, on the other hand, has rarely been associated with viral infection; the two known examples are polyomavirus, which was found to be hydroxylated on capsid proteins (Ludlow and Consigli, 1989), and the algal Paramecium bursaria Chlorella virus-1, which was shown to encode a prolyl-4-hydroxylase (Eriksson et al., 1999). Hydroxylation of prolines is a common modification in collagens, increasing helical stability by allowing formation of intermolecular hydrogen bonds. Failure to hydroxylate collagen leads to partial unfolding, destabilization of collagen fibrils and onset of a pathological condition known as scurvy (Mussini et al., 1967). The present disclosure has revealed that collagen prolyl hydroxylases modify ZIKV and DENV nascent chains to promote stabilization of E protein. Unincorporated E protein is subjected to ERAD, and KD of RPLP1 or AUP1, a lipid droplet protein, both result in increased degradation of E protein and reduced ZIKV and DENV production (Campos et al., 2017; Zhang et al., 2018). Additional work is needed to characterize the role of hydroxyproline in folding, oligomerization or stabilization of viral proteins. Nevertheless, infection is delayed in the absence of sufficient proline hydroxylation, suggesting that the shutoff of global translation could also serve to free up prolyl hydroxylases associated with collagen nascent chains.

Thus the present disclosure is the first-of-its-kind compendium of time-resolved polysome interactions during viral infection. Such an analysis provides meaningful insights into how viruses induce specialized translation states. A new biosynthetic pathway in flavivirus biology is revealed herein, and specific targets for antiviral intervention are provided in various embodiments of the present disclosure. The platform described herein is also be used for exploring co-translational interactions in other viruses to reveal otherwise hidden layers of virus biology.

In one embodiment, the present disclosure provides a method of treating or preventing an infection from a RNA virus in a subject comprising the steps of administering a therapeutic agent, wherein said therapeutic agent is an inhibitor of (i) viral protein translation and/or (ii) an inhibitor of viral replication to the subject, wherein said inhibitor is capable of inhibiting the function of one or more eukaryotic initiation factors associated with a viral polysome.

In another embodiment, a method of treating or preventing an infection from a RNA virus in a subject comprising the steps of administering an inhibitor of (i) viral protein folding and assembly and/or (ii) an inhibitor of viral replication to the subject, wherein said inhibitor is capable of inhibiting the function of one or more collagen prolyl hydroxylases and/or one or more collagen prolyl hydroxylase coenzymes is provided.

As used herein, to “treat” or “treating” means reducing the frequency with which symptoms of a disease are experienced by a patient. Treatment may be prophylactic (to prevent or delay the onset of the disease, or to prevent (e.g., “preventing”) the manifestation of clinical or subclinical symptoms thereof) or therapeutic suppression or alleviation of symptoms after the manifestation of the disease. The term “treat” includes the administration of the compounds or therapeutic agents of the present disclosure to (i) prevent or delay the onset of the symptoms, complications, or biochemical indicia of, (ii) alleviate the symptoms of, and/or (iii) inhibit or arrest the further development of, the disease, condition, or disorder. In one embodiment, a method as described herein of preventing an infection is provided prior to exposure to a flavivirus or prior to the subject entering a region that is prone to flaviviral infections.

RNA viruses, as used herein, include, but are not limited to, single-stranded RNA viruses, double-stranded RNA viruses, negative-sense and positive-sense, and ambisense RNA viruses. Exemplary RNA viruses as disclosed herein include members of the flaviviridae such as Dengue virus, Zika virus, Hepatitis C virus, West Nile virus, Yellow Fever virus, Japanese Encephalitis virus, Tick-borne Encephalitis, St-Louis Encephalitis; members of the picornaviridae such as poliovirus, coxsackievirus, rhinovirus, enterovirus 71 and 68, hepatitis A, FMD; members of the alphavirus family such as chickungunya and equine encephalitis; and members of the coronavirus family such as SARS, MERS and SARS-CoV2.

Except when noted, the terms “patient” or “subject” are used interchangeably and refer to mammals such as human patients and non-human primates, as well as veterinary subjects such as rabbits, rats, and mice, and other animals. Preferably, “patient” or “subject” refers to a human. In certain embodiments, a subject is an adult human. In certain embodiments, a subject is a child.

In various embodiments, the therapeutic agent includes, but is not limited to, a small molecule inhibitor, an antibody or function antibody derivate,

The eukaryotic initiation factor-4A (eIF4A) family consists of 3 closely related proteins EIF4A1, EIF4A2, and EIF4A3. EIF4A1 and EIF4A2 are required for the binding of 40S ribosomal subunits to mRNA. In addition these proteins are helicases that function to unwind double-stranded RNA.

In one embodiment, an inhibitor used in the methods described herein is rocaglamide (“RocA”) or other members of this family of compounds. Rocaglamide is a natural product which belongs to a class of molecules called flavaglines. Like other flavaglines, rocaglamide displays potent insecticidal, antifungal, anti-inflammatory and anticancer activities. Rocaglamide A (RocA) inhibits eukaryotic translation initiation by binding to the translation initiation factor eIF4A and converting it into a translational repressor.

In various embodiments and as described herein, the initiation factor is a RNA helicase. RNA helicases include, but are not limited to, DDX3X/Y, DDX46, DDX55, AQR, DDX23, DDX18, DDX21, DDX50, DDX39B, DHX9, and DDX6. In one embodiment, the small molecule inhibitor RK-33 is contemplated.

Prolyl hydroxylation is a post-translational modification that affects the structure, stability and function of proteins including collagen by catalyzing hydroxylation of proline to hydroxyproline through action of collagen prolyl hydroxylases 3 (C-P3H) and 4 (C-P4H). C-P3Hs—P3H1, P3H2 and P3H4 (nomenclature was amended according to approval by the HGNC symbols and names and Entrez database) (aka leucineproline-enriched proteoglycan (leprecan) 1 (Leprel), leprecan-like 1 (Leprel1), leprecan-like 2 (Leprel2)) and two paralogs Cartilage-Related Protein (CRTAP) and leprecan-like 4 (Leprel4) are found in humans. The C-P4Hs are tetrameric proteins comprising a variable a subunit, encoded by the P4HA1, P4HA2 and P4HA3 genes and a constant_{ft} subunit encoded by P4HB.

Diethyl-pythiDC (“pythiDC”) is an inhibitor of collagen prolyl 4-hydroxylases (CP4Hs). In one embodiment, pythiDC is used in the methods described herein. In other embodiments, pythiDC derivatives are contemplated, such as NOG, 24PDC, 25PDC, DHB, N-acylsulfonamide, 2-heterocyclic glycinamide, bipy, bipy45′DC, bipy55′DC, alpha-ketoglutarate, deferoxamine, pyoxDC, pyimDC (Vasta et al., ACS Chem. Biol., 2016, 11(1): 193-199). In other embodiments, EDHB (Ethyl 3,4-dihydroxybenzoate).

As described herein, combinations of the therapeutic agents, including the inhibitors described herein, can be used in combination therapies. In various embodiments, anti-viral small molecule, an inhibitor of a chaperone protein, small molecule inhibitor, an antibody or binding fragment thereof, an oligonucleotide, and a vector encoding an oligonucleotide can be administered alone or in combination. In this way, the therapeutic agent or second/third, etc therapeutic agent may include inhibitors of a Hsp70 chaperone, or an Hsp90 chaperone, or a cofactor of Hsp70 or Hsp90 or Hsp47.

“Combination therapy” embraces administration of therapeutic agents in a sequential manner, that is, wherein each therapeutic agent is administered at a different time, as well as administration of these therapeutic agents, or at least two of the therapeutic agents, in a substantially simultaneous manner. Sequential or substantially simultaneous administration of each therapeutic agent can be effected by any appropriate route as described herein, including, but not limited to, intranasal, inhalation (e.g., by nebulizer), buccal or sublingual, systemic routes, e.g., intravenous, intraperitoneal, enteric (including oral), intramuscular, subcutaneous, and transmucosal routes; and topical and transdermal routes.

Dosage regimens can be adjusted to provide the optimum desired response. For example, a single bolus can be administered, several divided doses can be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the active compound or compounds are well-known in the relevant art and would be understood to be encompassed by the skilled artisan once provided the teachings disclosed herein.

By the term “therapeutically effective amount,” as used herein, is meant an amount that when administered to a mammal, preferably a human, mediates a detectable therapeutic response compared to the response detected in the absence of the compound. A therapeutic response, such as, but not limited to, inhibition of and/or decreased infection or amelioration of one or more symptoms of viral infection, e.g., flavivirus infection, including, e.g., such methods as disclosed herein.

The skilled artisan would understand that the effective amount of the compound or composition administered herein varies and can be readily determined based on a number of factors such as the disease or condition being treated, the stage of the disease, the age and health and physical condition of the mammal being treated, the severity of the disease, the particular compound being administered, and the like.

In other embodiments of the present disclosure, a method of identifying a polysome-associated protein in a cell infected by a virus is provided. Such a method is not limited to RNA viruses; any virus may be used.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### EXAMPLES

Flaviviruses, including Dengue, Zika, and West Nile Virus, are mosquito-borne RNA viruses that pose a major threat to public health, with over a 100 million new infections each year. The three viruses are commonly associated with febrile disease, but in some cases can lead to life-threatening conditions e.g. microcephaly in unborn babies (Zika) and hemorrhagic fever in children and adults (Dengue). No effective vaccines or other therapeutics are currently available to prevent or treat infection with any of these viruses.

The following examples provide a proteomic screen from which host factors from infected cells that bind to polysomes and nascent chain producing viral proteins in cells infected with Dengue virus and Zika virus. It was found that, during infection of cultured human liver cells with either Zika or Dengue, several host factors bind preferentially to viral translation complex. As described herein, at least two factors can be targeted pharmacologically for therapeutic applications. First, eIF4A is specifically required for translation of viral proteins. Inhibition of this factor using drug RocA blocks viral replication with little toxicity. Second, five members of the collagen proline hydroxylase family are recruited to viral nascent chains, where they modify multiple proline sites. This is the first disclosure implicating collagen proline hydroxylases in the lifecycle of any virus. CP4H depletion by combined knockdown of P4HA1 and P4HA2 did not affect cell viability in culture but was associated with loss of proline hydroxylation on viral proteins and significantly lower production of infectious viral particles. A recently developed CP4H inhibitor, PythiDC can also inhibit virus replication. It was further found that the effect of CP4H depletion in infected cells is mediated by misprocessing of the viral polyprotein precursor, likely affecting the function of individual viral proteins and impairing the production of viral progeny.

Thus, the present disclosure reveals an unexpected dependence of Zika and Dengue viruses on specific translation factors and protein biogenesis factors that bind to polysomes translating viral proteins. In particular, host prolyl hydroxylase CP4H and eIF4A provide novel targets for antiviral interventions. Inhibitors of eIF4A and of ER prolyl hydroxylase can be used in the clinic to prevent and treat infection with Zika, Dengue, and possibly other viruses from related families, with minimal toxicity. As described herein, there are currently no effective vaccines or antivirals for the prevention or treatment of Zika and Dengue virus infection. Due to the acute nature of infection with these viruses, treatment duration is expected to be short. In influenza, another RNA virus, established antiviral treatment courses generally range from 1 to 7 days; for comparison, the average onset of scurvy, the main known outcome of CP4H inhibition, takes over 4 weeks to develop.

### Example 1

### RNA Virus Infection Induces Extensive Remodeling of Polysome Composition

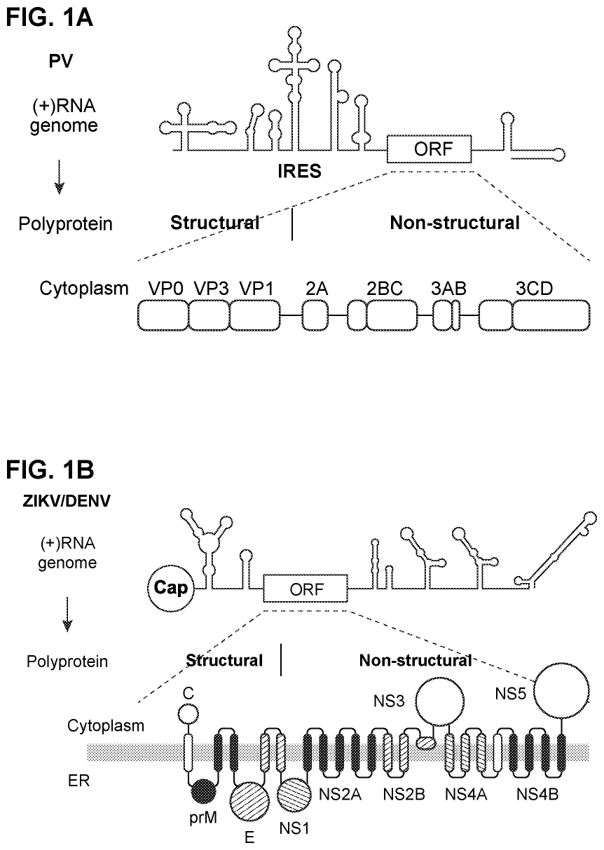

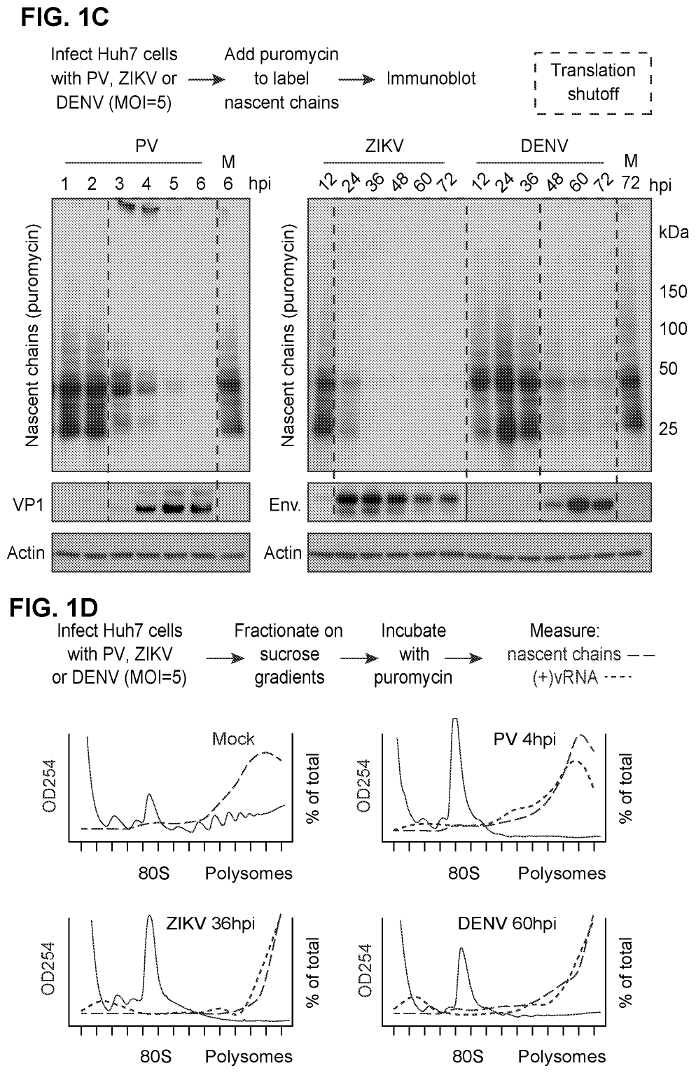

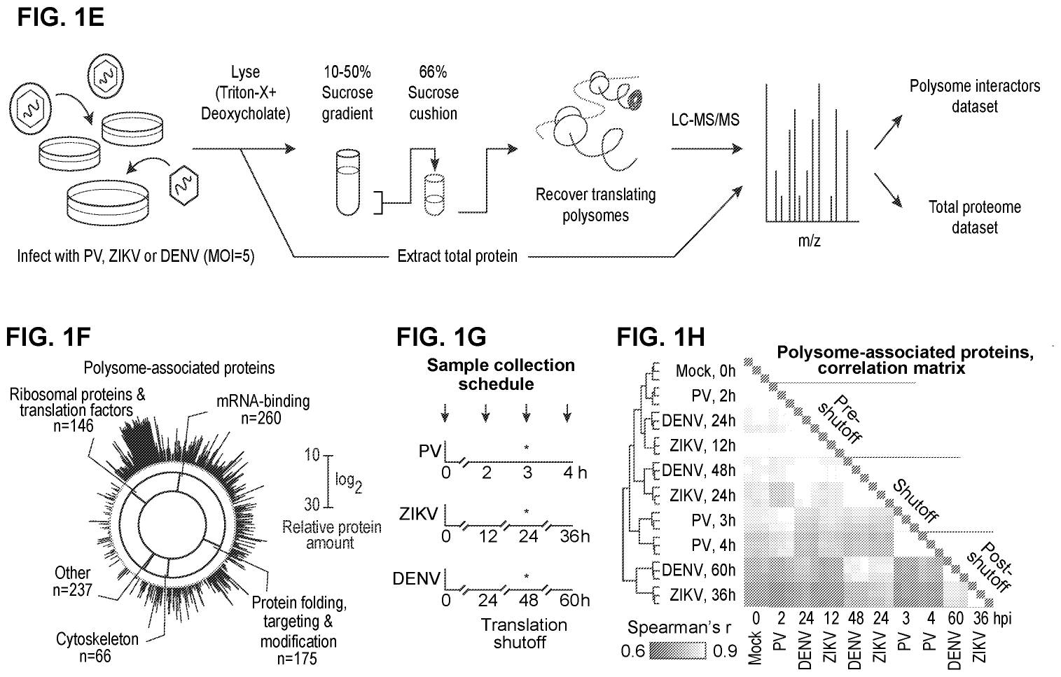

PV, a member of the Picornaviridae family, is translated in the host cytosol as a single polyprotein from a ˜7 kb plus-strand RNA genome with a 5′ IRES and a 3′ polyadenylate (poly-A) tail. Two viral proteases co- and post-translationally cleave the polyprotein into 10 discrete structural and nonstructural proteins (FIG. 1A). ZIKV and DENV, closely related members of the Flaviviridae family, have ˜11 kb plus-strand RNA genomes with short, capped 5′ untranslated regions (UTR) and highly structured 3′ UTRs with no poly-A tail. While ZIKV and DENV have a 5′ cap, they can still be translated under conditions that suppress cap-dependent initiation (28074025), although a canonical IRES has not been identified. Their polyproteins, which share a ˜70% homology at the amino acid level, are co-translationally translocated into the ER through multiple internal signal sequences and cleaved by both host and viral proteases into 10 discrete structural and nonstructural proteins (FIG. 1B).

To better understand the different synthetic strategies employed by these viruses, the effect of infection on global protein synthesis was monitored. Huh7 cells were infected with either PV-1 Mahoney, ZIKV PRVABC59 or DENV-2 strain 16681 at a multiplicity of infection (MOI) of 5. At select time-points during infection, puromycin was added to tissue culture media to label nascent polypeptides, and lysates were subjected to immunoblotting. Analysis of puromycylated peptides revealed that all three viruses induced a shutoff of protein synthesis, although the time course varied (FIG. 1C). Production of viral proteins increased as global translation decreased (FIG. 1C), suggesting a switch to noncanonical mechanisms of translation. To confirm that viral RNA can efficiently engage ribosomes after this global shutoff, cells were infected as before and fractionated lysates on 10-50% sucrose gradients. Fractions were collected, incubated with puromycin to label nascent polypeptides, and assayed for the presence of both puromycylated peptides and plus-strand viral RNA by immunodetection and qPCR, respectively. While little-to-no polysome traces were observed in infected cells (FIG. 1D, gray), consistent with translation shutoff, both viral RNA (blue) and nascent polypeptides (orange) co-sedimented in the heaviest fractions of the gradients, supporting the presence of viral polysomes. In contrast, viral polymerases were predominantly detected in the low-density fractions of the gradients, suggesting that under these fractionation conditions, polysomes can be separated from replication compartments.

Next, polysomes were extracted for proteomic analysis as shown in FIG. 1E. Cells were simultaneously infected with each of the three viruses at an MOI of 5, fractionated lysates as before, collected polysome-containing fractions and pelleted them on a 66% sucrose cushion. Isolated polysomes and matching total proteome samples were digested and analyzed by liquid chromatography tandem mass-spectrometry (LC-MS/MS). MS analysis of polysome samples, collected in triplicates, detected over 1,000 proteins with high inter-replicate reproducibility. Most abundant were components of the core ribosome, reflecting about 2-fold enrichment over the total proteome, followed by proteins that associate with either the RNA template or the nascent chain product (FIG. 1F). Sampling times were chosen based on the individual time course of each virus, to reflect similar biosynthetic stages i.e. before, during and after shutoff of translation (FIG. 1G). Most proteins were detected and quantified in all samples. Viral polypeptides were not detected in the earliest timepoints for each virus, but later increased in abundance to about 1:1 ratio with ribosomal proteins, indicating that viral RNA occupies an increasingly larger proportion of polysomes as infection progresses. The increase in proportion of viral polysomes coincided with translation shutoff and was associated with extensive changes in polysome composition, as reflected by Spearman's correlation coefficients (FIG. 1H).

### Example 2

### Polysome Composition Dynamics are Driven by Pan-Viral and Virus-Specific Rearrangement of Co-Translational Networks

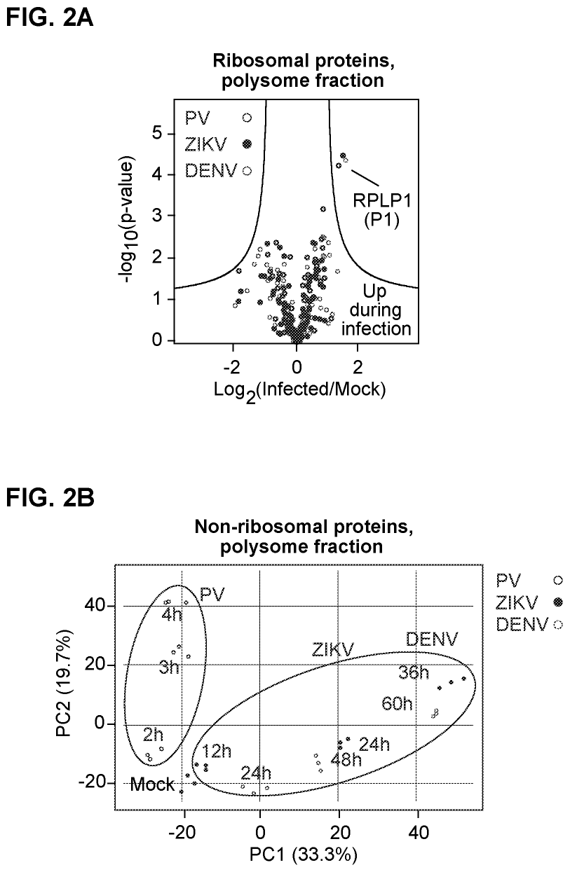

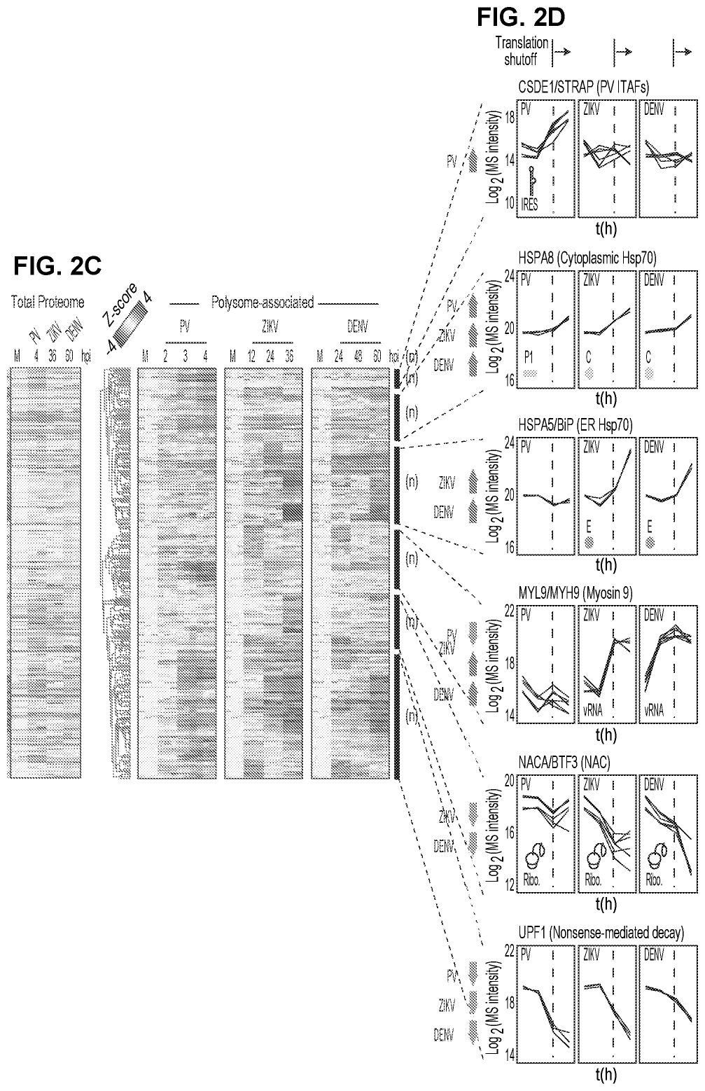

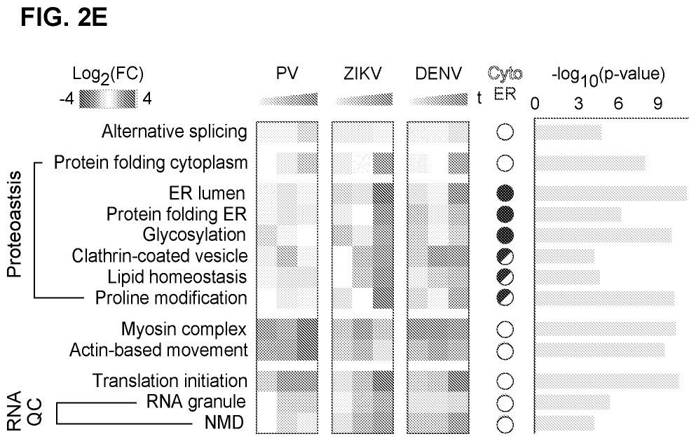

To determine whether the core ribosome particle is remodeled during infection, the abundance of ribosomal proteins in polysome fractions during peak infection with each virus versus mock was compared. The stoichiometry of ribosomal proteins was maintained throughout, with one exception: all three viruses induced an up to 4-fold increase in the polysome content of the 60S subunit acidic stalk protein P1 (FIG. 2A). The ribosomal P stalk, comprised of uL10 (P0), P1 and P2, is a flexible protrusion that recruits elongation factors to the ribosome and mediates stress signaling. In contrast to other ribosomal proteins, P1 and P2 are not assembled in the nucleus but constantly shuttle between mature ribosomes and a cytoplasmic pool (Tsurugi and Ogata, 1985). P1 can be depleted without major effects on global protein synthesis (Perucho et al., 2014) and was recently shown to be required for translation of ZIKV and DENV (Campos et al., 2017), although the specific mechanism is not understood. This data suggests that these viral RNAs may be preferentially translated by ribosomes harboring P1.

Next, changes in non-ribosomal proteins was explored. Pairwise comparisons of polysome interactors between mock infection and each of the infected timepoints revealed increasing dynamics of both recruitment and dissociation during infection. Principal component analysis (PCA) of polysome interactors minus ribosomal proteins showed a clear divide between PV and either ZIKV or DENV (FIG. 2B), indicating that polysome dynamics induced by ZIKV and DENV are similar but distinct from those induced by PV. Unsupervised hierarchical clustering of the full dataset revealed 6 major groups based on temporal patterns of association (FIG. 2C). Overall, these dynamics were not secondary to degradation or de novo synthesis, as steady-state levels of polysome interactors did not change significantly (FIG. 2C), despite viral polyproteins occupying a significant fraction of the proteome. The 6 groups consist of proteins that are either recruited to or depleted from polysomes in some or all the viruses, and a representative example is given for each (FIG. 2D). CSDE1 (Unr) and its binding partner STRAP (UnrIP), an RNA-binding complex that stimulates translation from PV IRES, was only recruited to polysomes translating PV, while the cytosolic and ER Hsp70s (HSPA8 and HSPAS/BiP), which bind viral structural proteins, showed pan-viral and flavivirus-specific increase in polysome association, respectively. In contrast, both subunits of the nascent chain associated complex (NAC) were drastically displaced from polysomes translating ZIKV and DENV, but not PV, possibly to allow co-translational targeting to the ER. Finally, helicase UPF1, which regulates RNA surveillance as part of its role in nonsense mediated decay and antiviral signaling, was depleted from polysomes across all three viruses.

An analysis of functional annotations revealed a specific enrichment of alternative splicing factors on polysomes translating PV, consistent with known involvement of splicing factors such as PCBP, PTBP and SRSF7 (9G8) in translation initiation from PV IRES (REFs). It further showed pan-viral recruitment of cytosolic chaperones and flaviviral-specific recruitment of ER-resident factors involved in folding and other biosynthetic pathways previously implicated in flavivirus infection e.g. glycosylation, lipid homeostasis and peptidylprolyl modification (FIG. 2E). Actomyosin trafficking networks were specifically recruited to polysomes translating ZIKV and DENV, but depleted from polysomes translating PV. Finally, translation initiation and RNA surveillance factors were depleted off polysomes for all three viruses.

### Example 3

### Infection is Associated with Cleavage or Sequestration of RNA Surveillance Factors, Resulting in their Depletion from Polysomes

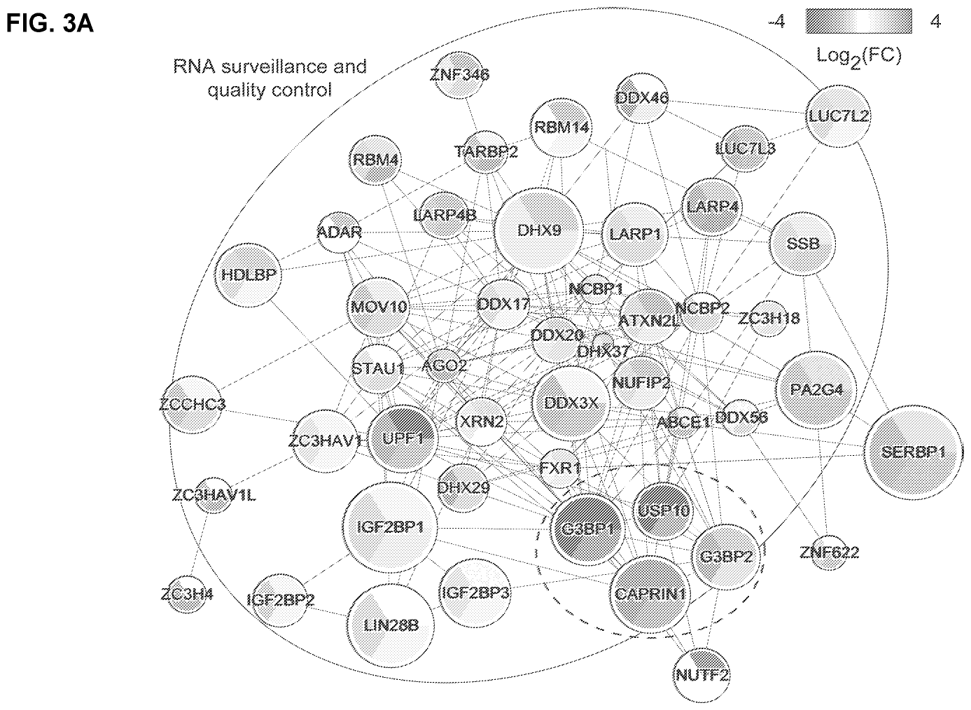

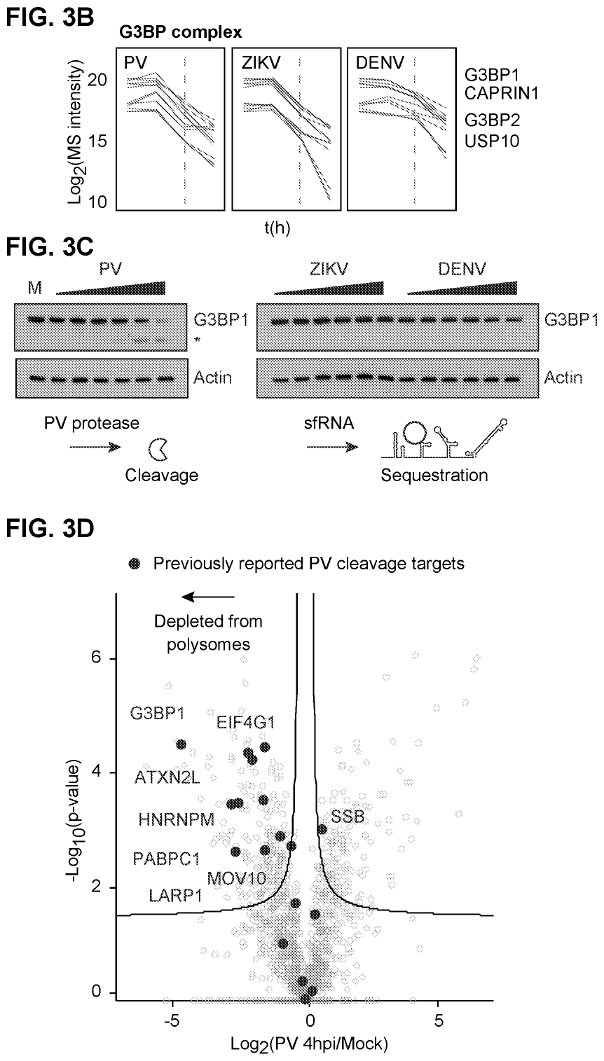

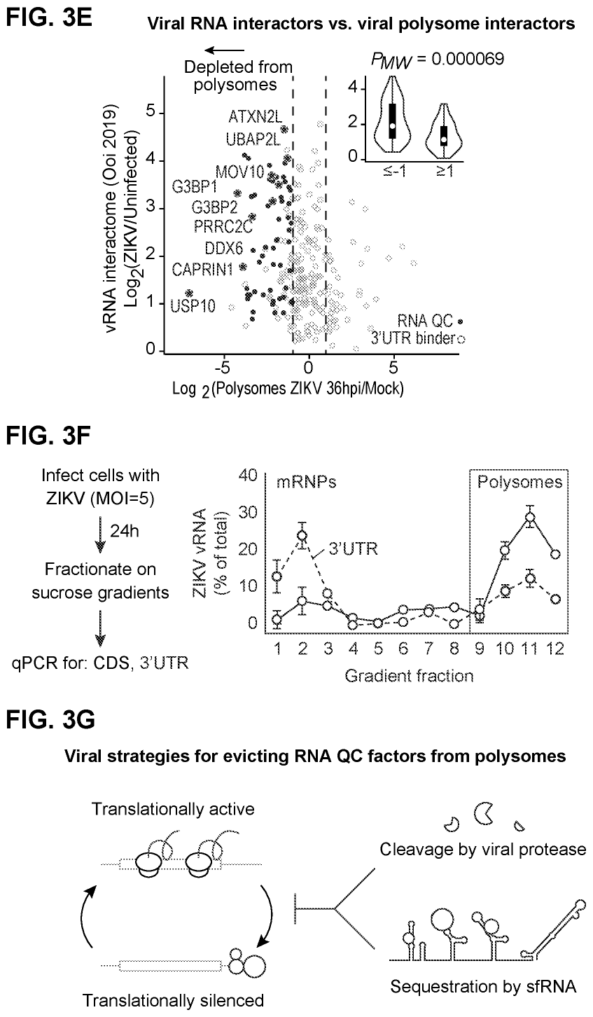

Protein-protein interaction networks were generated to visualize each functional category of host factors implicated in infection. The network for RNA surveillance and quality control (FIG. 3A) showed pan-viral depletion of multiple factors with known antiviral functions, including G3BP stress granule factors (FIG. 3B), which are known to bind and sequester viral RNA away from ribosomes to prevent its translation. Many viruses have evolved to disrupt such functions, including G3BP1 cleavage by PV (White et al., 2007) and sequestration by DENV subgenomic RNA (Bidet et al., 2014). Indeed, G3BP1 cleavage was only observed for PV, but not ZIKV or DENV (FIG. 3C), and this was associated with reduced co-sedimentation of G3BP1 with translating polysomes. Other known targets of PV cleavage were searched, and found that many of them, including ATXN2L, HNRNPM and LARP1, were depleted from polysomes during PV infection (FIG. 3D), suggesting that such depletion may be the result of cleavage. In contrast, during ZIKV and DENV infection, cleavage by host exonuclease XRN1 generates noncoding 3′UTR fragments called subgenomic flaviviral RNAs (sfRNA), which were shown to sponge certain host factors (Bidet et al., 2014; Moon et al., 2015). To explore whether ZIKV and DENV sfRNA may sequester antiviral factors away from polysomes, a dataset of ZIKV and DENV RNA pulldown MS (Ooi et al., 2019) was analyzed. While most RNA interactors were also identified as polysome associated, high affinity interactors were predominantly depleted from polysomes in both ZIKV (FIG. 3E) and DENV, pointing towards pervasive vRNA interactions that occur off ribosomes. Such interactors include bonafide ZIKV and DENV 3′UTR binders e.g. G3BP1/2, ATXN2L, MOV10 and DDX6, suggesting that competition between full length vRNA and sfRNA may shift these factors away from polysomes. Indeed, when polysome association of coding versus 3′UTR vRNA during ZIKV infection were compared, 3′UTR RNA was found to be about 4-fold more abundant in the free mRNA fractions of sucrose gradients as compared to CDS RNA (FIG. 3F).

### Example 4

### Helicase eIF4A1 Affects Both Translation and Replication of Flaviviruses

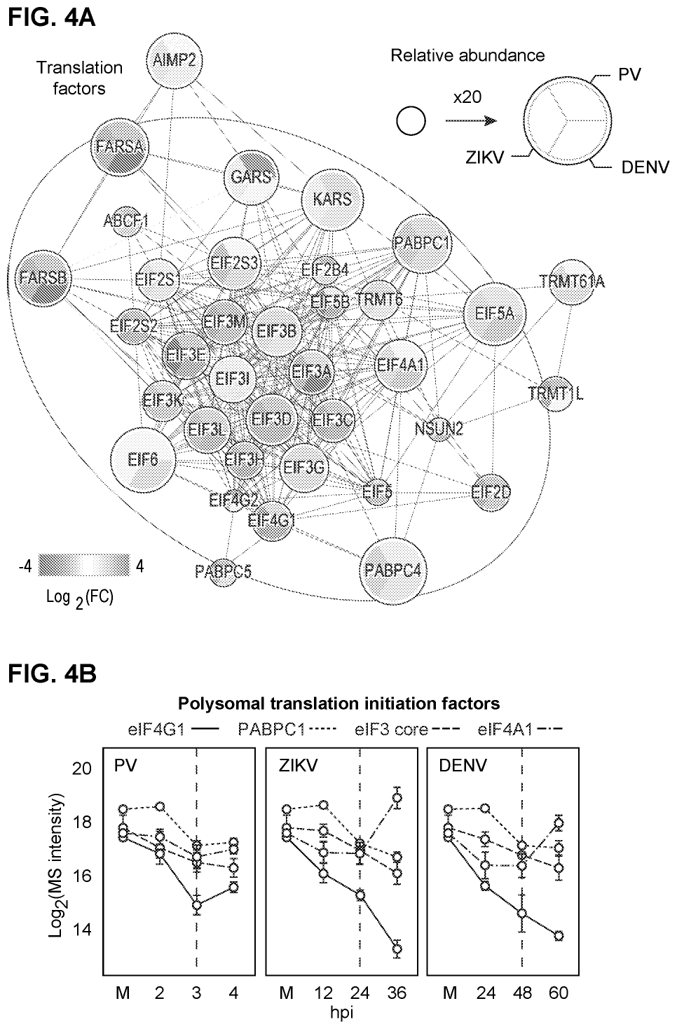

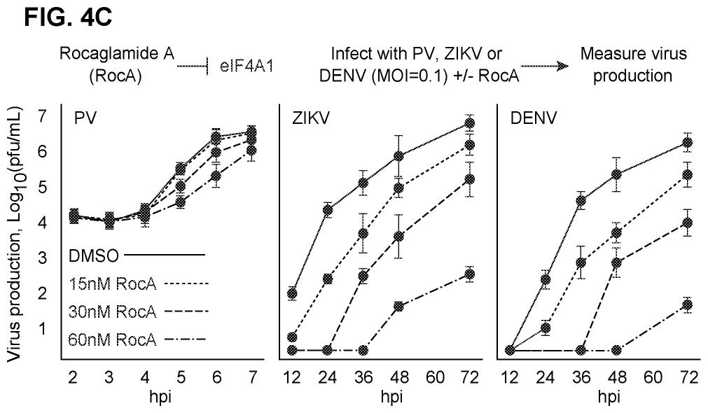

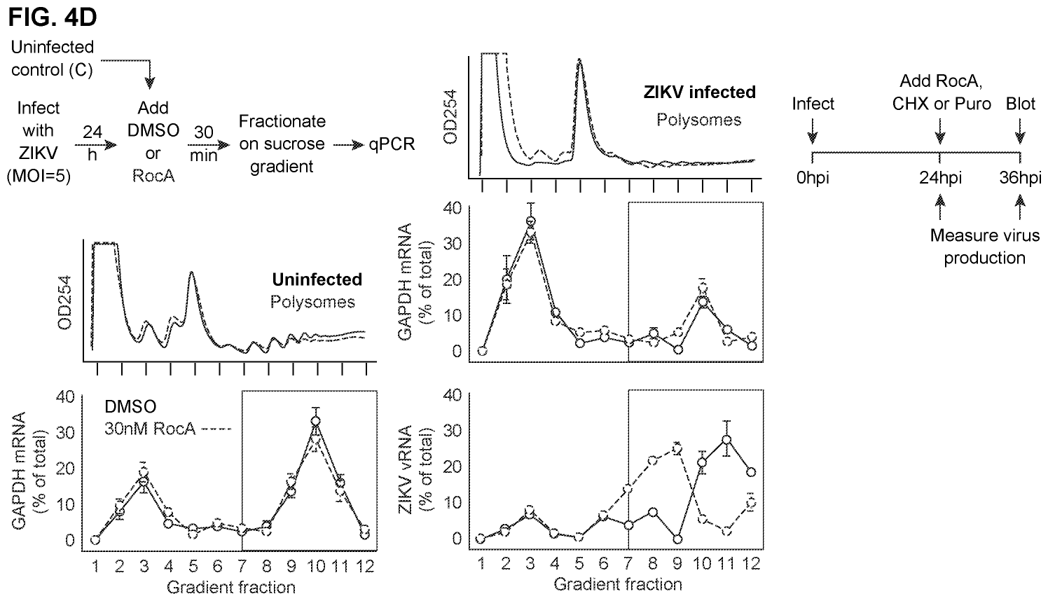

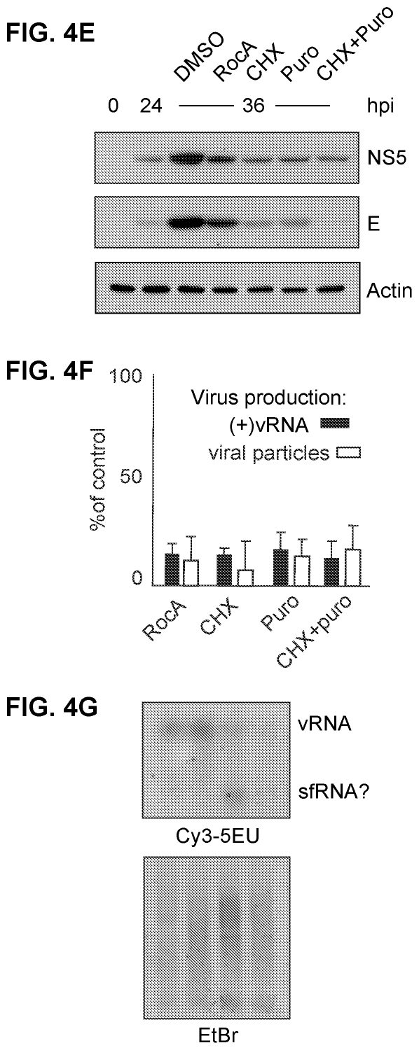

Factors involved in translation were analyzed. Protein-protein interaction analysis showed that most translation initiation factors were depleted across all three viruses (FIG. 4A). Because the open reading frames (ORFs) of PV, ZIKV and DENV are longer than the average cellular ORF (Balchin et al., 2016), they could potentially harbor a higher than average number of elongating ribosomes. The polysome data represent the sum of all initiating and elongating ribosomes, and the observed decrease in translation initiation factors could therefore reflect a lower proportion of initiating ribosomes on a longer than average RNA. Although the ratio of 40S to 60S ribosomal proteins in polysome fractions did drop by about 20-30% during infection with either virus, some translation factors were depleted by more than 90%, indicating that additional selective mechanisms must be in place. One such mechanism involves tRNA synthetases; while most did not change in polysome association, phenylalanine tRNA synthetase A and B (FARSA/B) were depleted by about 10-fold for each of the three viruses, while glycine aminoacyl tRNA synthetase (GARS) was recruited specifically to polysomes translating PV, consistent with its stimulatory role in translation initiation from PV IRES (Andreev et al., 2012). Interestingly, components that mediate the interaction between ribosomes and RNA, including eIF4G1, poly-A binding protein (PABPC1) and the core subunits of the eIF3 complex were depleted off polysomes in all three viruses (FIG. 4A). The two modules of eIF3, namely the octamer and yeast-like core, showed distinct patterns of dissociation, suggesting that the complex may undergo rearrangements in response to viral infection. Both eIF4G1 and PABPC1 are cleaved by many viruses, including PV, to prevent formation of closed-loop polysome structures that stimulate translation of cellular mRNA (Stern-Ginossar et al., 2019). Both were similarly depleted off polysomes during infection with any of the viruses, but peptide-level analysis revealed that PABPC1 was uniformly depleted, while the C-terminal cleavage fragment of eIF4G1 was specifically retained on PV polysomes, consistent with its in vitro stimulatory function in PV translation (Ohlmann et al., 1996).

Strikingly, polysome association of helicase eIF4A1, which unwinds secondary RNA structures in 5′ UTRs of cellular mRNAs, increased during infection with ZIKV and DENV but not PV (FIG. 4B). To determine whether this increase reflects hyperdependence of ZIKV and DENY, Rocaglamide A (RocA), a specific eIF4A1 inhibitor with minimal effects on cell viability in the low nanomolar range, was tested. When infection was launched in the presence of RocA, a drastic dose-dependent decrease in virus production was observed for ZIKV and DENV but not PV (FIG. 4C). To confirm that this effect was mediated by reduced translation initiation, cells were infected with ZIKV for 24 h then added RocA for 30 min. Lysates were fractionated on 10-50% sucrose gradients and assayed gradient fractions for GAPDH mRNA and ZIKV plus-strand RNA by qPCR. A 30-min RocA treatment did not affect the distribution of GAPDH mRNA in either mock or ZIKV-infected cells, but resulted in a shift of ZIKV RNA to lighter fractions (FIG. 4D), consistent with inhibition of translation initiation. Nevertheless, this suggested that RocA delayed rather than completely inhibited synthesis of viral proteins. To address that, cells were infected with ZIKV and at 24 h added RocA, cycloheximide (CHX) or puromycin (Puro) to inhibit protein synthesis, and analyzed viral protein, RNA and particles at 36 hpi. As compared to CHX or Puro, RocA had a mild inhibitory effect on accumulation of either NS5 or E (FIG. 4E). However, all three had similar effects on accumulation of viral RNA or particles (FIG. 4F). RocA is known to function by stably clamping eIF4A1 to its target sites on RNA (Iwasaki et al., 2016), and it is therefore hypothesized that it may directly inhibit ZIKV replication. Active crude replication complexes were isolated from ZIKV-infected cells and performed in vitro replication in the presence of a uridine analogue (5EU) to label newly synthesized RNA. ZIKV replication was reduced in the presence of either RocA or 2′CMA, an NS5 inhibitor (FIG. 4G). This proteomic data further showed that multiple other RNA helicases are recruited to viral polysomes, including DDX3X/Y, DDX46, DDX55, AQR, DDX23, DDX18, DDX21, DDX50, DDX39B, DHX9, DDX6, and it is tempting to speculate that some could be attractive targets for antiviral drug development.

### Example 5

### Polysome-Associated Chaperones Regulate Multiple Events in the Biosynthesis of Viral Proteins

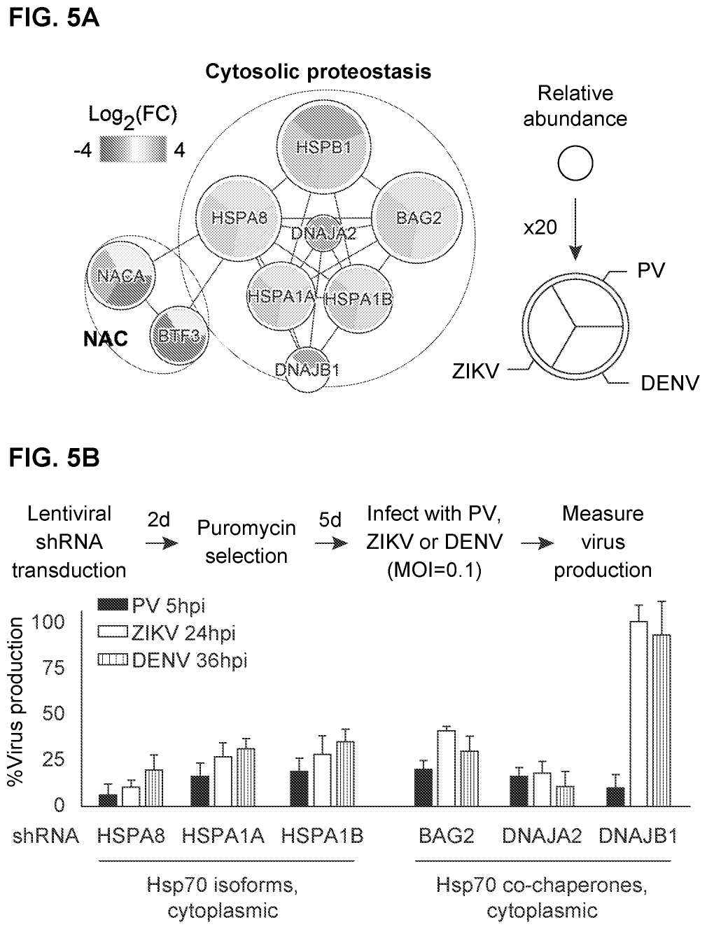

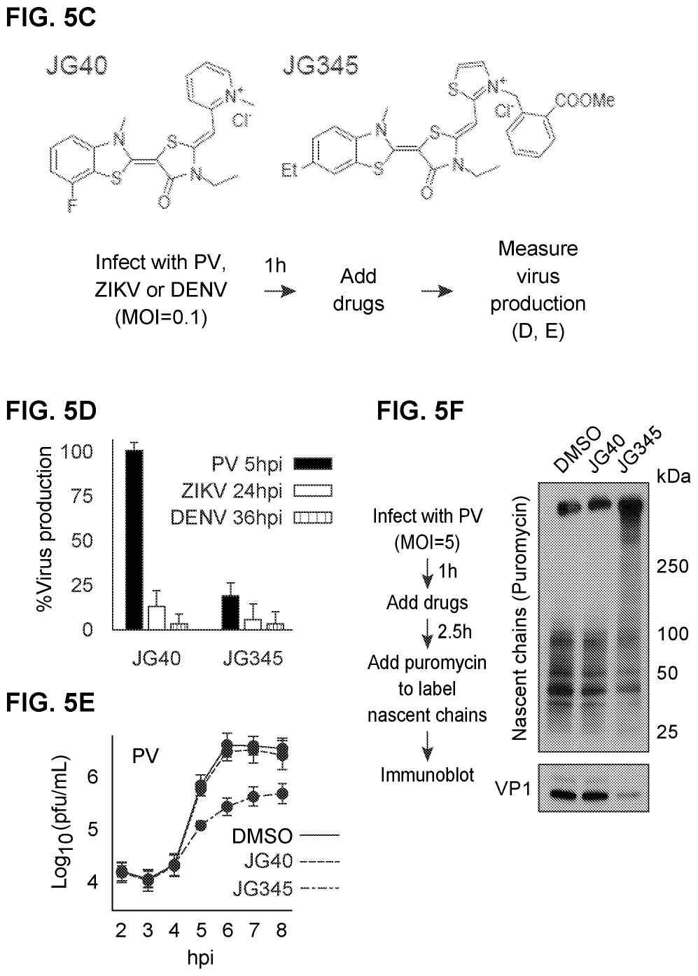

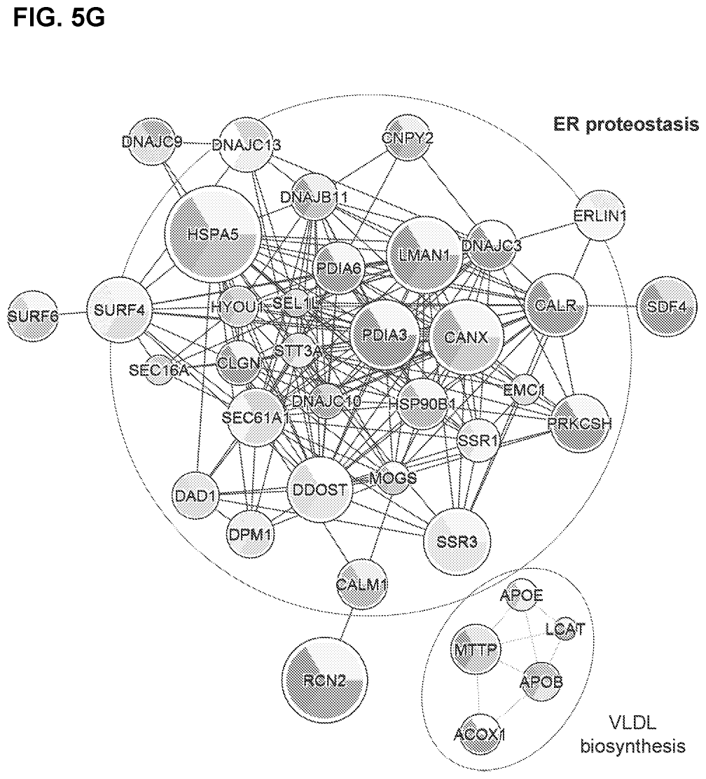

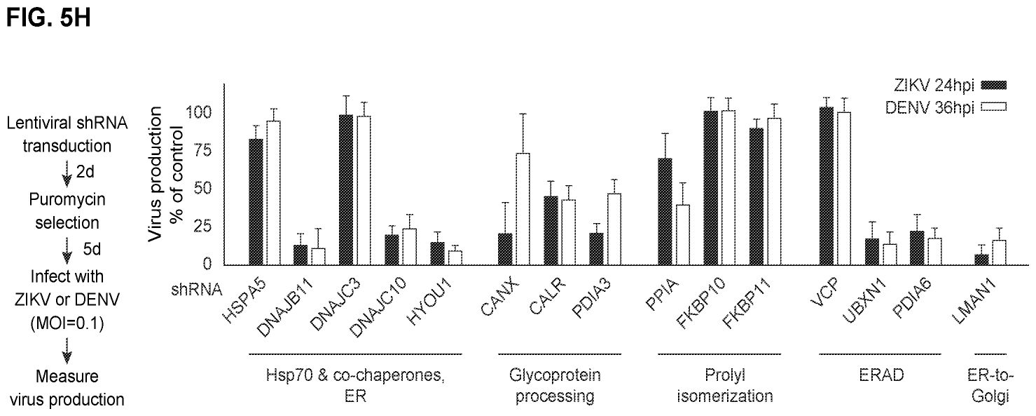

Multiple members of the cytosolic Hsp70 chaperones were found to be recruited to polysomes across all three viruses (FIG. 5A), including constitutive and inducible isoforms (HSPA8, HSPA1A/B) as well as co-chaperones DNAJA2 and BAG2, which promote folding and substrate release by Hsp70. DNAJB1, which also promotes folding by Hsp70, was uniquely recruited to PV polysomes. In agreement with these observations, knockdown of the above factors inhibited production of all three viruses, except for DNAJB1, which was only needed for PV production (FIG. 5B). Next, two allosteric inhibitors against Hsp70 (FIG. 5C) were tested, and it was found that, at non-toxic concentrations, both inhibited ZIKV and DENV but only one inhibited PV (FIG. 5D). This inhibition was dose-dependent and maintained throughout the infectious cycle (FIG. 5E). While the roles of Hsp70s in ZIKV and DENV infection were rigorously studied and found to involve entry, RNA replication and virion biogenesis (Taguwa et al., 2015, 2019), less is known about Hsp70 in PV. It was previously reported that Hsp70 interacts co-translationally with PV capsid precursor, P1 (Macejak and Sarnow, 1992), which consists of multiple oligomerization domains and is therefore prone to aggregation (Korant, 1973). In these analyses, puromycylated nascent chain aggregates were detected between 3 and 4 h of infection with PV (FIG. 1C). To determine whether aggregation begins pre- or post-release from ribosomes, lysates from mock- and PV-infected cells were digested with RNase I, then fractionated the lysates on sucrose gradients, incubated with puromycin to label nascent polypeptides, and assayed for the presence puromycylated peptides by immunoblot. If elongating nascent chains begin to aggregate through intramolecular interactions, RNase treatment should yield 80S monosomes; however, if multiple nascent chains aggregate through intermolecular interactions, the same treatment should result in ribosomes migrating as disomes or higher order polysomes. RNase I treatment of PV- but not mock-infected lysates resulted in ribosome-nascent chain aggregates sedimenting as monosomes, suggesting that intramolecular aggregation occurs as nascent chains are elongating. To test whether Hsp70 protects nascent PV chains from such aggregation, cells were infected with PV in the absence or presence of Hsp70 inhibitors and, at 3.5 hpi, added puromycin to label nascent chains. In the presence of JG345 but not JG40, an increase was observed in aggregated puromycylated peptides and a decrease in the levels of fully processed VP1 capsid protein (FIG. 5F). Taken together, these results support a role for Hsp70 in binding PV capsid precursor and preventing premature co-translational oligomerization that impairs production of viral progeny.

While cytosolic Hsp70s were recruited by all viruses, both subunits of NAC (NACA/BTF3), which is stably bound to ribosomes to promote translation and protein folding, were specifically displaced off polysomes during ZIKV and DENV infection. NAC is known to be displaced by binding of the signal recognition complex (SRP) to nascent signal sequences but is thought to reassociate and assist with ER targeting of the complex (Zhang et al., 2012). Although SRP association did not change, an overall increase was observed in association of ER luminal factors following depletion of NAC from ZIKV and DENV polysomes (FIG. 5F). Cumulative distribution analyses found that polysome interactors annotated as “ER part” were first depleted during the early steps of ZIKV and DENV infection, only to be recruited again at a later stage, consistent with a previous report that DENV infection preferentially hijacks polysomes that are already ER-associated (Reid et al., 2018). Such ER resident proteins include Hsp90 (Grp94), components of the calnexin-calreticulin glycoprotein folding cycle, mediators of calcium-dependent processing, factors involved in lipid droplet formation, prolyl isomerases, and proteins involved in ER-associated degradation and ER-to-Golgi trafficking (FIG. 5G). Knockdowns of select members of each functional category resulted in reduced ZIKV and DENV titers (FIG. 5H), confirming their importance to the viral life cycle.

### Example 6

### Collagen Prolyl Hydroxylases Promote ZIKV and DENY Infection

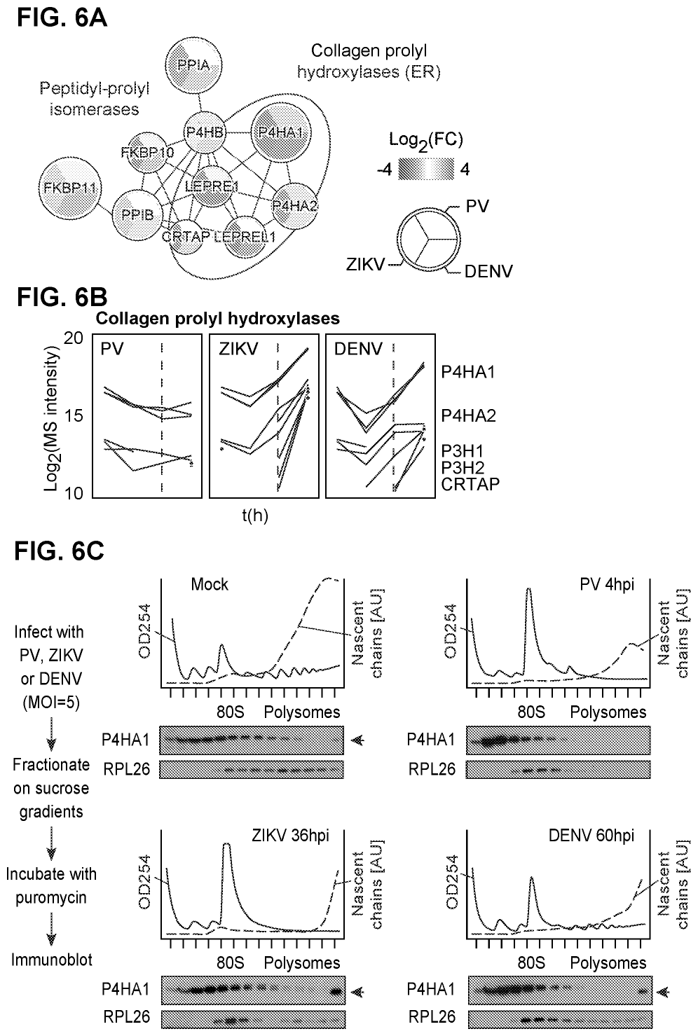

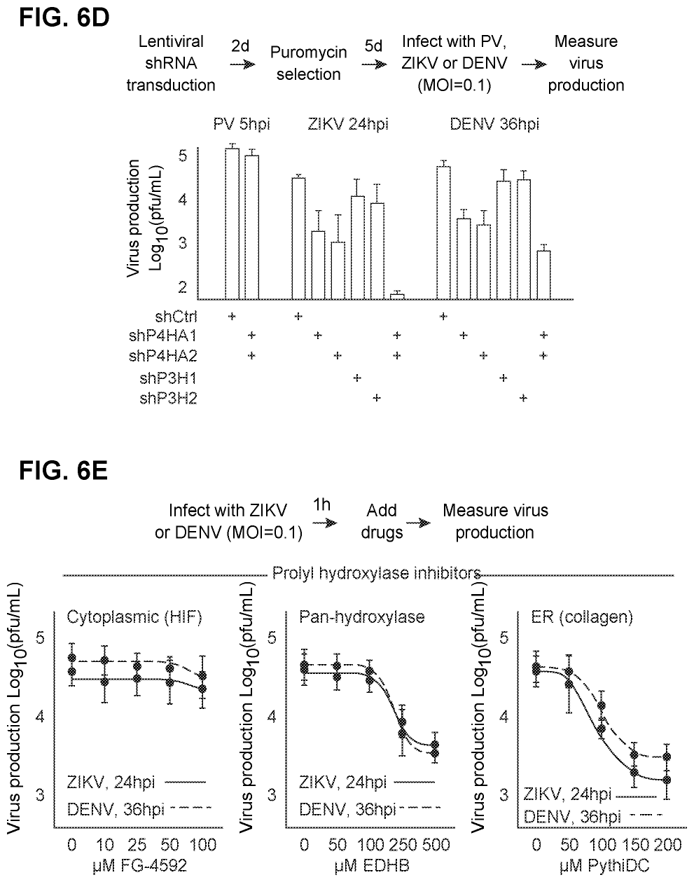

Most of the pathways listed above were previously implicated in flavivirus biology, but no role was known for proline hydroxylation. Five collagen prolyl hydroxylases (cP4H) were found to be recruited to polysomes translating ZIKV and DENV, but not PV, including four enzymes that mediate hydroxylation at position 4 (P4HA1/2) or 3 (P3H1/2) and a structural scaffold (CRTAP) (FIG. 6A,B). As collagen proline hydroxylation can occur co-translationally (Kirk et al., 1987), the association of hydroxylases with viral polysomes was analyzed. To this end, cells were infected with PV, ZIKV or DENV, fractionated lysates on 10-50% sucrose gradients, incubated gradient fractions with puromycin to label nascent polypeptides and analyzed by immunoblot. Co-sedimentation of puromycylated peptides with P4HA1, the main collagen prolyl hydroxylase in most cell types (Annunen et al., 1998), decreased during PV infection and increased during ZIKV and DENV infection (FIG. 6C). Next, cell lines were generated stably expressing shRNAs against collagen prolyl hydroxylases and assayed the effects on virus particle production. Knockdown of either P4HA1 or P4HA2 reduced ZIKV and DENV titers, but not PV, and a combined knockdown of both P4HA1 and P4HA2 inhibited virus production even further (FIG. 6D). The combined knockdown delayed production of ZIKV and DENV, but titers eventually recovered to WT levels when infection was allowed to proceed up to 72 h. Minimally toxic small molecule inhibitors against ER, but not cytoplasmic proline hydroxylases had similar though weaker inhibitory effects on both ZIKV and DENV (FIG. 6E), confirming that cP4H enzymes promote flavivirus infection.

### Example 7

### Proline Hydroxylation of ZIKV Polypeptide Stabilizes Envelope Protein

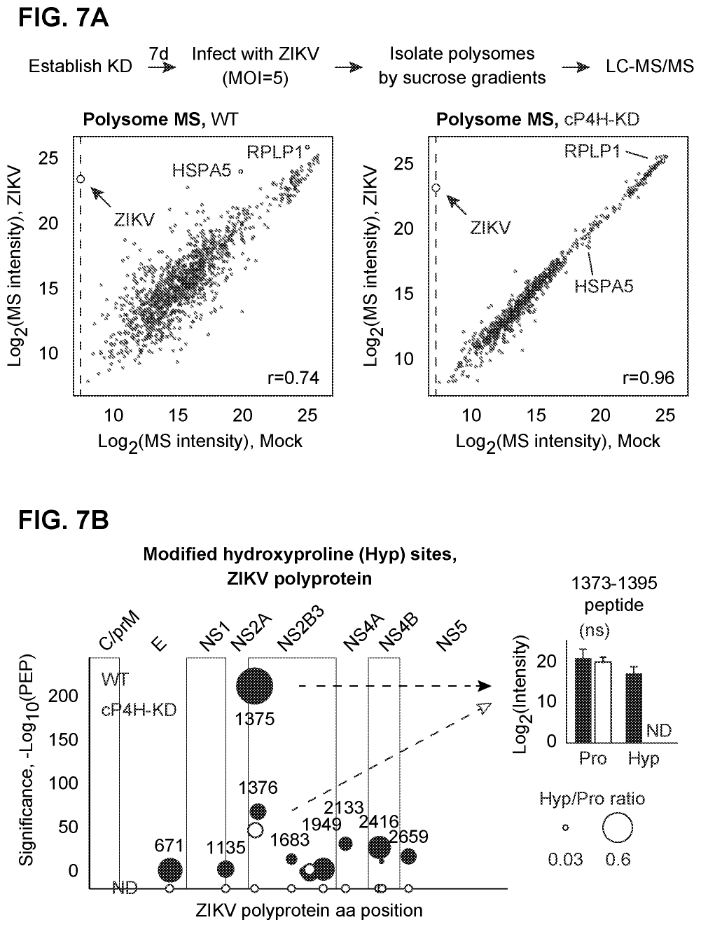

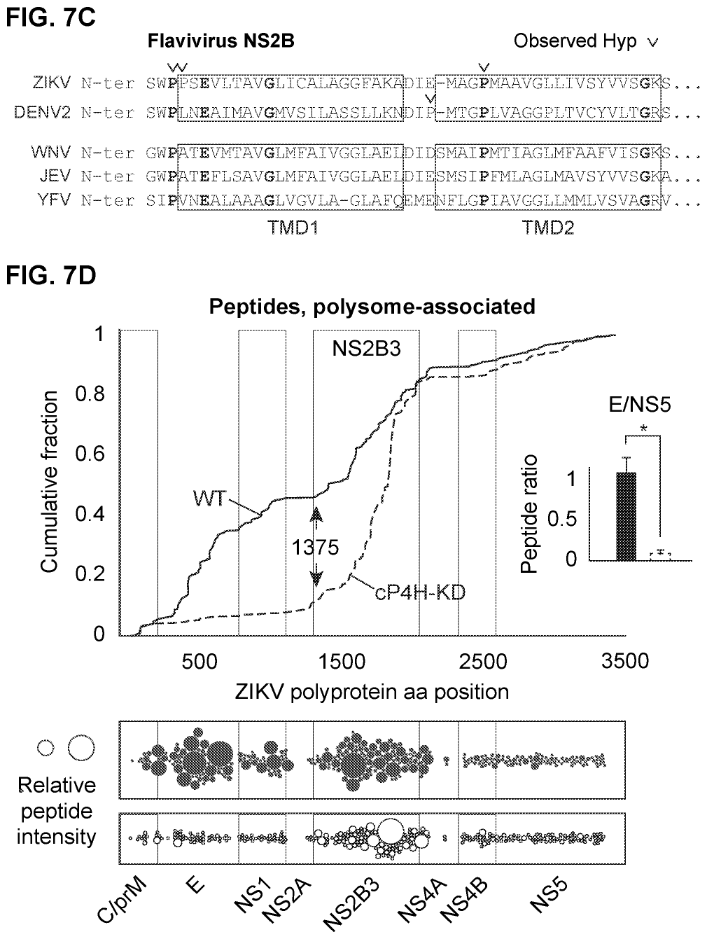

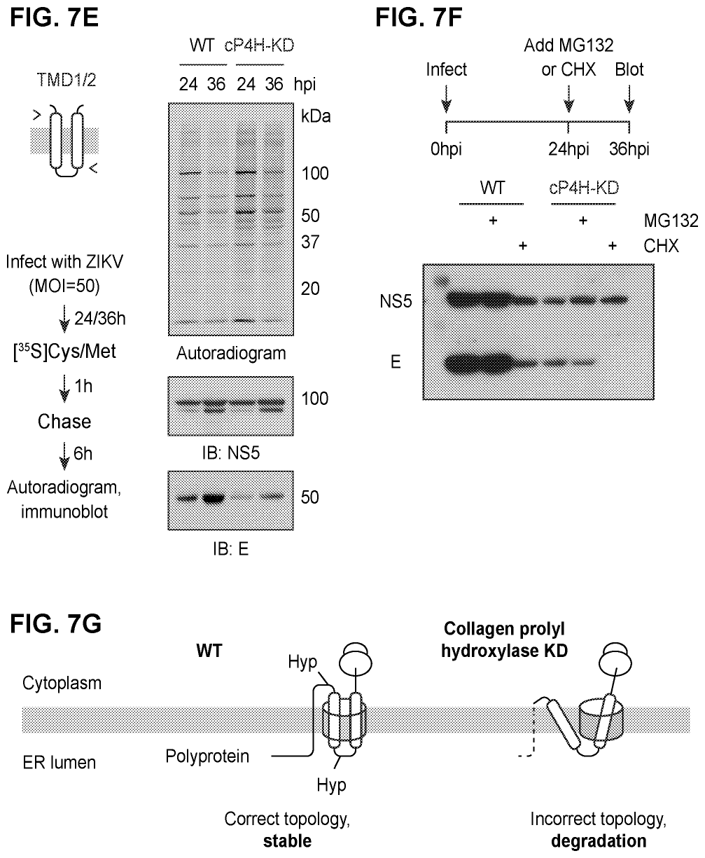

The effect of cP4H-KD on kinetics of ZIKV production and ZIKV-induced shutoff of translation was tested. Compared to WT, depletion of cP4H delayed translation shutoff. To determine whether this was associated with differences in polysome remodeling, cP4H-KD cells were infected with ZIKV for 48 h, isolated polysomes as before and analyzed their composition by MS. Compared to WT, polysome-associated P4HA1/2 levels in the KD cells were lower by orders of magnitude. Strikingly, no polysome remodeling was observed in cP4H-KD cells by 48 h despite similar levels of ZIKV proteins (FIG. 7A), although significant changes in polysome composition were detected in WT cells as early as 24 hpi. This suggested that, in the absence of cP4H, viral protein level or function was insufficient to promote polysome remodeling. Next, the proteomic dataset was searched for evidence of proline hydroxylation. Hydroxyproline sites were manually validated based on a typical m/z shift of oxygen addition (+15.99) in multiple b and y fragment ions, representing N- and C-ter fragments, respectively (FIG. S7D). Multiple prolines were found to be hydroxylated on ZIKV polyprotein in WT cells infected for 24 and 36 h, with the most abundant modifications affecting up to 60% of a given site (FIG. 7B, black). Most of these sites were not detected in cP4H-KD cells infected for 48 h (FIG. 7B, orange). A similar level of unmodified peptides was detected in both cell lines, suggesting that the reduced detection of hydroxyproline sites was not due to lower peptide abundance (FIG. 7B, bar graph). A similar search revealed the presence of hydroxyproline sites on the DENV polyprotein, albeit with lower frequency and statistical significance. Highly modified sites in ZIKV NS2B were found to be conserved across multiple flaviviruses, despite low local sequence conservation (FIG. 7C). To determine whether cP4H-KD had any effect on polyprotein stoichiometry, individual peptides were analyzed from the polysomes MS. Peptides originating from the N-terminal part of the polyprotein, upstream to the major hydroxyproline site in NS2B, were drastically reduced in KD cells, lowering the ratio of E-to-NS5 (FIG. 7D). To test whether this was related to a synthetic defect, WT and cP4H-KD cells were infected with ZIKV and metabolically labeled newly-synthesized proteins at 24 and 36 hpi using radioactive amino acids, followed by a 6-h chase. Overall translation output was not reduced in cP4H KD cells compared to WT controls (FIG. 7E, top), and an immunoblot of the same samples confirmed that E levels were significantly lower in the KD cells while NS5 levels were equivalent (FIG. 7E, bottom). To test whether this was a result of degradation, cells were infected with ZIKV for 24 h and then added either CHX or MG132 for another 12 h. While NS5 and E levels were similarly affected by CHX or MG132 in the WT cells, E was strongly destabilized in the KD cells (FIG. 7F). Thus, ZIKV polyprotein processing requires proline hydroxylation by cP4H to achieve correct topology and that in the absence of cP4H, E protein is more rapidly degraded (FIG. 7G).

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

### REFERENCES

Andreev, D. E., Hirnet, J., Terenin, I. M., Dmitriev, S. E., Niepmann, M., and Shatsky, I. N. (2012). Glycyl-tRNA synthetase specifically binds to the poliovirus IRES to activate translation initiation. Nucleic Acids Res. 40, 5602-5614.
Annunen, P., Autio-Harmainen, H., and Kivirikko, K. I. (1998). The novel type II prolyl 4-hydroxylase is the main enzyme form in chondrocytes and capillary endothelial cells, whereas the type I enzyme predominates in most cells. J. Biol. Chem. 273, 5989-5992.
Aviner, R., and Frydman, J. (2020). Proteostasis in Viral Infection: Unfolding the Complex Virus-Chaperone Interplay. Cold Spring Harb. Perspect. Biol. 12.
Aviner, R., Hofmann, S., Elman, T., Shenoy, A., Geiger, T., Elkon, R., Ehrlich, M., and Elroy-Stein, 0. (2017). Proteomic analysis of polyribosomes identifies splicing factors as potential regulators of translation during mitosis. Nucleic Acids Res. 45, 5945-5957.
Balchin, D., Hayer-Hartl, M., and Hartl, F. U. (2016). In vivo aspects of protein folding and quality control. Science 353, aac4354.
Bidet, K., Dadlani, D., and Garcia-Blanco, M. A. (2014). G3BP1, G3BP2 and CAPRIN1 are required for translation of interferon stimulated mRNAs and are targeted by a dengue virus non-coding RNA. PLoS Pathog. 10, e1004242.
Biedenkopf, N., Lange-Grünweller, K., Schulte, F. W., Weißer, A., Müller, C., Becker, D., Becker, S., Hartmann, R. K., and Grünweller, A. (2017). The natural compound silvestrol is a potent inhibitor of Ebola virus replication. Antiviral Res. 137, 76-81.
Campos, R. K., Wong, B., Xie, X., Lu, Y.-F., Shi, P.-Y., Pompon, J., Garcia-Blanco, M. A., and Bradrick, S. S. (2017). RPLP1 and RPLP2 Are Essential Flavivirus Host Factors That Promote Early Viral Protein Accumulation. J. Virol. 91.
Choe, Y.-J., Park, S.-H., Hassemer, T., Korner, R., Vincenz-Donnelly, L., Hayer-Hartl, M., and Hartl, F. U. (2016). Failure of RQC machinery causes protein aggregation and proteotoxic stress. Nature 531, 191-195.
Desmet, E. A., Anguish, L. J., and Parker, J. S. L. (2014). Virus-mediated compartmentalization of the host translational machinery. MBio 5, e01463-14.
Elgner, F., Sabino, C., Basic, M., Ploen, D., Grunweller, A., and Hildt, E. (2018). Inhibition of Zika Virus Replication by Silvestrol. Viruses 10.
Eriksson, M., Myllyharju, J., Tu, H., Hellman, M., and Kivirikko, K. I. (1999). Evidence for 4-hydroxyproline in viral proteins. Characterization of a viral prolyl 4-hydroxylase and its peptide substrates. J. Biol. Chem. 274, 22131-22134.
Fan, Y.-H., Nadar, M., Chen, C.-C., Weng, C.-C., Lin, Y.-T., and Chang, R.-Y. (2011). Small noncoding RNA modulates japanese encephalitis virus replication and translation in trans. Virol. J. 8, 492.
Friedrich, S., Engelmann, S., Schmidt, T., Szczepankiewicz, G., Bergs, S., Liebert, U. G., Kümmerer, B. M., Golbik, R. P., and Behrens, S.-E. (2018). The Host Factor AUF1 p45 Supports Flavivirus Propagation by Triggering the RNA Switch Required for Viral Genome Cyclization. J. Virol. 92.
Genuth, N. R., and Barna, M. (2018). Heterogeneity and specialized functions of translation machinery: from genes to organisms. Nat. Rev. Genet. 19, 431-452.
Henss, L., Scholz, T., Grünweller, A., and Schnierle, B. S. (2018). Silvestrol Inhibits Chikungunya Virus Replication. Viruses 10.
Imami, K., Milek, M., Bogdanow, B., Yasuda, T., Kastelic, N., Zauber, H., Ishihama, Y., Landthaler, M., and Selbach, M. (2018). Phosphorylation of the Ribosomal Protein RPL12/uL11 Affects Translation during Mitosis. Mol. Cell 72, 84-98.e9.
Isken, O., Baroth, M., Grassmann, C. W., Weinlich, S., Ostareck, D. H., Ostareck-Lederer, A., and Behrens, S.-E. (2007). Nuclear factors are involved in hepatitis C virus RNA replication. RNA 13, 1675-1692.
Iwasaki, S., Floor, S. N., and Ingolia, N. T. (2016). Rocaglates convert DEAD-box protein eIF4A into a sequence-selective translational repressor. Nature 534, 558-561.
Jaafar, Z. A., and Kieft, J. S. (2019). Viral RNA structure-based strategies to manipulate translation. Nat. Rev. Microbiol. 17, 110-123.
Kirk, T. Z., Evans, J. S., and Veis, A. (1987). Biosynthesis of type I procollagen. Characterization of the distribution of chain sizes and extent of hydroxylation of polysome-associated pro-alpha-chains. J. Biol. Chem. 262, 5540-5545.
Korant, B. D. (1973). Cleavage of poliovirus-specific polypeptide aggregates. J. Virol. 12, 556-563.
Landry, D. M., Hertz, M. I., and Thompson, S. R. (2009). RPS25 is essential for translation initiation by the Dicistroviridae and hepatitis C viral IRESs. Genes Dev. 23, 2753-2764.
Lee, A. S.-Y., Burdeinick-Kerr, R., and Whelan, S. P. J. (2013). A ribosome-specialized translation initiation pathway is required for cap-dependent translation of vesicular stomatitis virus mRNAs. Proc. Natl. Acad. Sci. U.S.A. 110, 324-329.
Ludlow, J. W., and Consigli, R. A. (1989). Hydroxyproline in the major capsid protein VP1 of polyomavirus. J. Virol. 63, 2881-2884.
Macejak, D. G., and Sarnow, P. (1992). Association of heat shock protein 70 with enterovirus capsid precursor P1 in infected human cells. J. Virol. 66, 1520-1527.
Majzoub, K., Hafirassou, M. L., Meignin, C., Goto, A., Marzi, S., Fedorova, A., Verdier, Y., Vinh, J., Hoffmann, J. A., Martin, F., et al. (2014). RACK1 controls RES-mediated translation of viruses. Cell 159, 1086-1095.
Mizuno, C. M., Guyomar, C., Roux, S., Lavigne, R., Rodriguez-Valera, F., Sullivan, M. B., Gillet, R., Forterre, P., and Krupovic, M. (2019). Numerous cultivated and uncultivated viruses encode ribosomal proteins. Nat. Commun. 10, 752.
Moon, S. L., Dodd, B. J. T., Brackney, D. E., Wilusz, C. J., Ebel, G. D., and Wilusz, J. (2015). Flavivirus sfRNA suppresses antiviral RNA interference in cultured cells and mosquitoes and directly interacts with the RNAi machinery. Virology 485, 322-329.
Mussini, E., Hutton, J. J., and Udenfriend, S. (1967). Collagen proline hydroxylase in wound healing, granuloma formation, scurvy, and growth. Science 157, 927-929.
Ohlmann, T., Rau, M., Pain, V. M., and Morley, S. J. (1996). The C-terminal domain of eukaryotic protein synthesis initiation factor (eIF) 4G is sufficient to support cap-independent translation in the absence of eIF4E. EMBO J. 15, 1371-1382.
Ooi, Y. S., Majzoub, K., Flynn, R. A., Mata, M. A., Diep, J., Li, J. K., van Buuren, N., Rumachik, N., Johnson, A. G., Puschnik, A. S., et al. (2019). An RNA-centric dissection of host complexes controlling flavivirus infection. Nat. Microbiol. 4, 2369-2382.
Peel, M., and Scribner, A. (2013). Cyclophilin inhibitors as antiviral agents. Bioorg. Med. Chem. Lett. 23, 4485-4492.
Perucho, L., Artero-Castro, A., Guerrero, S., Ramon y Cajal, S., LLeonart, M. E., and Wang, Z.-Q. (2014). RPLP1, a crucial ribosomal protein for embryonic development of the nervous system. PLoS One 9, e99956.
Reid, D. W., Campos, R. K., Child, J. R., Zheng, T., Chan, K. W. K., Bradrick, S. S., Vasudevan, S. G., Garcia-Blanco, M. A., and Nicchitta, C. V (2018). Dengue Virus Selectively Annexes Endoplasmic Reticulum-Associated Translation Machinery as a Strategy for Co-opting Host Cell Protein Synthesis. J. Virol. 92.
Sanford, T. J., Mears, H. V, Fajardo, T., Locker, N., and Sweeney, T. R. (2019). Circularization of flavivirus genomic RNA inhibits de novo translation initiation. Nucleic Acids Res. 47, 9789-9802.
Shiber, A., Doring, K., Friedrich, U., Klann, K., Merker, D., Zedan, M., Tippmann, F., Kramer, G., and Bukau, B. (2018). Cotranslational assembly of protein complexes in eukaryotes revealed by ribosome profiling. Nature 561, 268-272.
Simsek, D., Tiu, G. C., Flynn, R. A., Byeon, G. W., Leppek, K., Xu, A. F., Chang, H. Y., and Barna, M. (2017). The Mammalian Ribo-interactome Reveals Ribosome Functional Diversity and Heterogeneity. Cell 169, 1051-1065.e18.
Slaine, P. D., Kleer, M., Smith, N. K., Khaperskyy, D. A., and McCormick, C. (2017). Stress Granule-Inducing Eukaryotic Translation Initiation Factor 4A Inhibitors Block Influenza A Virus Replication. Viruses 9.
Stern-Ginossar, N., Thompson, S. R., Mathews, M. B., and Mohr, I. (2019). Translational Control in Virus-Infected Cells. Cold Spring Harb. Perspect. Biol. 11.
Sweeney, T. R., Abaeva, I. S., Pestova, T. V, and Hellen, C. U. T. (2014). The mechanism of translation initiation on Type 1 picornavirus IRESs. EMBO J. 33, 76-92.
Taguwa, S., Maringer, K., Li, X., Bernal-Rubio, D., Rauch, J. N., Gestwicki, J. E., Andino, R., Fernandez-Sesma, A., and Frydman, J. (2015). Defining Hsp70 Subnetworks in Dengue Virus Replication Reveals Key Vulnerability in Flavivirus Infection. Cell 163, 1108-1123.
Taguwa, S., Yeh, M.-T., Rainbolt, T. K., Nayak, A., Shao, H., Gestwicki, J. E., Andino, R., and Frydman, J. (2019). Zika Virus Dependence on Host Hsp70 Provides a Protective Strategy against Infection and Disease. Cell Rep. 26, 906-920.e3.
Tsurugi, K., and Ogata, K. (1985). Evidence for the exchangeability of acidic ribosomal proteins on cytoplasmic ribosomes in regenerating rat liver. J. Biochem. 98, 1427-1431.
White, J. P., Cardenas, A. M., Marissen, W. E., and Lloyd, R. E. (2007). Inhibition of cytoplasmic mRNA stress granule formation by a viral proteinase. Cell Host Microbe 2, 295-305.
Yoshikawa, H., Larance, M., Harney, D. J., Sundaramoorthy, R., Ly, T., Owen-Hughes, T., and Lamond, A. I. (2018). Efficient analysis of mammalian polysomes in cells and tissues using Ribo Mega-SEC. Elife 7.
Zhang, J., Lan, Y., Li, M. Y., Lamers, M. M., Fusade-Boyer, M., Klemm, E., Thiele, C., Ashour, J., and Sanyal, S. (2018). Flaviviruses Exploit the Lipid Droplet Protein AUP1 to Trigger Lipophagy and Drive Virus Production. Cell Host Microbe 23, 819-831.e5.
Zhang, Y., Berndt, U., Golz, H., Tais, A., Oellerer, S., Wolfle, T., Fitzke, E., and Rospert, S. (2012). NAC functions as a modulator of SRP during the early steps of protein targeting to the endoplasmic reticulum. Mol. Biol. Cell 23, 3027-3040.

What is claimed is:

1. A method of treating or preventing an infection from a RNA virus in a subject comprising the steps of administering a therapeutic agent, wherein said therapeutic agent is an inhibitor of (i) viral protein translation and/or (ii) an inhibitor of viral replication to the subject, wherein said inhibitor is capable of inhibiting the function of one or more eukaryotic initiation factors associated with a viral polysome.

2. A method of treating or preventing an infection from a RNA virus in a subject comprising the steps of administering an inhibitor of (i) viral protein folding and assembly and/or (ii) an inhibitor of viral replication to the subject, wherein said inhibitor is capable of inhibiting the function of one or more collagen prolyl hydroxylases and/or one or more collagen prolyl hydroxylase coenzymes.

3. The method of claim 1 wherein said one or more eukaryotic initiation factor is selected from the group consisting of eukaryotic initiation factor 4A1 (eIF4A1) and eukaryotic initiation factor 4A2 (eIF4A2).

4. The method of claim 2 wherein said one or more collagen prolyl hydroxylase is a collagen prolyl 3-hydroxylase (CP3H) selected from the group consisting of prolyl 3-hydroxylase 1 (P3H1), prolyl 3-hydroxylase 2 (P3H2), and prolyl 3-hydroxylase 3 (P3H3).

5. The method of claim 2 wherein said one or more collagen prolyl hydroxylase is a collagen prolyl 4-hydroxylase (CP4H) selected from the group consisting of prolyl 4-hydroxylase subunit alpha-1 (P4HA1), prolyl 4-hydroxylase subunit alpha-2 (P4HA2) and prolyl 4-hydroxylase subunit alpha-3 (P4HA3).

6. The method of claim 2 wherein said one or more collagen prolyl hydroxylase coenzymes is selected from the group consisting of cartilage associated protein (CRTAP) and synaptonemal complex 65 (Sc65/P3H4).

7. The method of any of the preceding claims wherein said RNA virus is a flavivirus

8. The method of claim 7 wherein the flavivirus is selected from the group consisting of Zika virus, Dengue virus, West Nile virus, Yellow Fever virus and Japanese Encephalitis virus.

9. The method of any of the preceding claims wherein said inhibitor is selected from the group consisting of a small molecule inhibitor, an antibody or binding fragment thereof, an oligonucleotide, and a vector encoding an oligonucleotide.

10. The method of claim 9 wherein said inhibitor is an oligonucleotide selected from the group consisting of a small inhibitory RNA (siRNA), a microRNA (miRNA), and a short hairpin RNA (shRNA).

11. The method of claim 9 wherein said inhibitor is a vector that encodes an oligonucleotide of claim 10, a nuclease and/or a guide RNA (gRNA).

12. The method of claim 3 wherein said eukaryotic initiation factor is eukaryotic initiation factor 4A1 (eIF4A1).

13. The method of claim 12 wherein said inhibitor is rocalgamide (RocA).

14. The method of claim 5 wherein said inhibitor is pythiDC.

15. The method of any of the preceding claims further comprising administering a second therapeutic agent.

16. The method of claim 15 wherein the second therapeutic agent is selected from the group consisting of an anti-viral small molecule, an inhibitor of a chaperone protein, small molecule inhibitor, an antibody or binding fragment thereof, an oligonucleotide, and a vector encoding an oligonucleotide.

17. The method of claim 16 wherein said oligonucleotide is a small inhibitory RNA (siRNA), a microRNA (miRNA), and a short hairpin RNA (shRNA).

18. The method of claim 16 wherein said vector encodes an oligonucleotide of claim 17, a nuclease and/or a guide RNA (gRNA).

19. The method of claim 16 wherein the second therapeutic agent inhibits the function of a Hsp70 chaperone or an Hsp90 chaperone, or a cofactor of Hsp70 or Hsp90 or Hsp47.

20. The method of claim 15 wherein the second therapeutic agent is selected from the group consisting of JG40, JG345, Apoptozole, PIFITHRIN-Mu, 115-7c, MAL3-101 (Hsp70), AK778, Co1003, BMS-986263/NDL02-s0201 (Hsp47); geldanamycin, radicicol, derrubone, ganetespib, celastrol, novobiocin, VER49009, AT13387, PU3, PUH71, PUWS13 (Hsp90); AUY922, (Hsp90) VER155008, JG98, JG13, JG48, YM-01, YM-08 MKT-077, and PES-CI (Hsp70)

21. The method of claim 15 wherein the second therapeutic agent is selected from the group consisting of an inhibitor is capable of inhibiting the function of one or more eukaryotic initiation factors associated with a viral polysome, one or more collagen prolyl hydroxylases and/or one or more collagen prolyl hydroxylase coenzymes.

22. The method of any of claims 1-12 wherein the therapeutic agent is administered prior to exposure and/or infection of said RNA virus.

23. The method of claim 22 further comprising administering a second therapeutic agent, wherein said second therapeutic agent is administered.

24. The method of any of the preceding claims wherein said subject is a human subject.

25. A method of inhibiting replication of a RNA virus in a cell comprising the steps of administering a therapeutic agent, wherein said therapeutic agent is an inhibitor of (i) viral protein translation and/or (ii) an inhibitor of viral replication to the subject, wherein said inhibitor is capable of (i) inhibiting the function of one or more eukaryotic initiation factors associated with a viral polysome or (ii) inhibiting the function of one or more collagen prolyl hydroxylases and/or one or more collagen prolyl hydroxylase coenzymes.

26. A method of inhibiting translation of viral proteins in a cell infected with a RNA virus comprising the steps of administering a therapeutic agent, wherein said therapeutic agent is an inhibitor of (i) viral protein translation and/or (ii) an inhibitor of viral replication to the subject, wherein said inhibitor is capable of (i) inhibiting the function of one or more eukaryotic initiation factors associated with a viral polysome or (ii) inhibiting the function of one or more collagen prolyl hydroxylases and/or one or more collagen prolyl hydroxylase coenzymes.

27. A method of inhibiting prolyl hydroxylation in a cell infected with a RNA virus comprising the steps of administering a therapeutic agent, wherein said therapeutic agent is an inhibitor of (i) viral protein translation and/or (ii) an inhibitor of viral replication to the subject, wherein said inhibitor is capable of inhibiting the function of one or more collagen prolyl hydroxylases and/or one or more collagen prolyl hydroxylase coenzymes.

28. A method of preventing or inhibiting viral-induced remodeling of a polysome in a cell comprising the steps of administering a therapeutic agent, wherein said therapeutic agent is an inhibitor of (i) viral protein translation and/or (ii) an inhibitor of viral replication to the subject, wherein said inhibitor is capable of (i) inhibiting the function of one or more eukaryotic initiation factors associated with a viral polysome or (ii) inhibiting the function of one or more collagen prolyl hydroxylases and/or one or more collagen prolyl hydroxylase coenzymes.

29. The method of any one of claims 25-28 wherein a second therapeutic agent is administered.

30. A method of identifying a polysome-associated protein in a cell infected by a virus comprising the steps of:
(a) obtaining a sample of cells;
(b) infecting said cells of (a) with a virus;
(c) isolating polysomes from infected cells of (b); and
(d) analyzing protein composition of said polysomes.


SEQUENCE LISTING (5 sequences)

SEQ ID NO: 1 - protein, 43 aa, Artificial Sequence
  Synthetic
SWPPSEVLTAVGLCALAKADIEMAGPMAAVGLLIVSYVVSGKS

SEQ ID NO: 2 - protein, 48 aa, Artificial Sequence
  Synthetic
SWPLNEAIMAVGMVSILASSLLKNDIPMTGPLVAGGPLTVCYVLTGRS

SEQ ID NO: 3 - protein, 49 aa, Artificial Sequence
  Synthetic
GWPATEVMTAVGLMFAIVGGLAELDIDSMAIPMTIAGLMFAAFVISGKS

SEQ ID NO: 4 - protein, 49 aa, Artificial Sequence
  Synthetic
GWPATEFLSAVGLMFAIVGGLAELDIESMSIPFMLAGLMAVSYVVSGKA

SEQ ID NO: 5 - protein, 49 aa, Artificial Sequence
  Synthetic
SIPVNEALAAAGLVGVLAGLAFQEMENFLGPIAVGGLLMMLVSVAAGRV